(19) United States
(12) Patent Application Publication
(10) Pub. No.: US 2024/0110220 A1
(43) Pub. Date: Apr. 4, 2024

(54) DETECTING BETA-LACTAMASE ENZYME ACTIVITY

(71) Applicants: COMMISSARIAT A L'ENERGIE ATOMIQUE ET AUX ENERGIES ALTERNATIVES, PARIS (FR); UNIVERSITE PARIS-SACLAY, GIF-SUR-YVETTE (FR)

(72) Inventors: Herve VOLLAND, GIF-SUR-YVETTE (FR); Christian MOGUET, GIF-SUR-YVETTE (FR); Thierry NAAS, LE KREMLIN-BICETRE (FR)

(21) Appl. No.: 18/262,523

(22) PCT Filed: Jan. 25, 2022
(86) PCT No.: PCT/FR2022/050133

§ 371 Date: Jul. 21, 2023

(30) Foreign Application Priority Data
Jan. 26, 2021 (FR) FR2100708

(51) Int. Cl. C12Q 1/34; C12Q 1/18

(52) U.S. Cl. CPC C12Q 1/34; C12Q 1/18; C12Y 305/02006

(57) ABSTRACT

It is essential to have efficient, simple, quick and transportable tools for reliably identifying bacteria that are multiresistant to antibiotics, more specifically extended spectrum β-lactamase (ESBL)-producing Enterobacteriaceae, which are the most widespread among Enterobacteriaceae. The present invention meets this requirement through its ease of use and its speed. The invention is based on detecting the enzyme activity of β-lactam hydrolysis using an antibody capable of discriminating between the intact form of the β-lactam ring of a β-lactam and its hydrolysis product. This antibody can be used in kits and methods enabling for rapidly detecting (in less than one hour), without using expensive equipment (a small strip visible to the naked eye), the presence of bacteria producing penicillin-type, plasmid-mediated or hyper-produced AmpC enzymes, of ESBL or carbapenemase from colonies or in a sample.

### SUMMARY OF THE INVENTION

It is essential to have efficient, simple, quick and transportable tools for reliably identifying bacteria that are multiresistant to antibiotics, more specifically extended spectrum β-lactamase (ESBL)-producing Enterobacteriaceae, which are the most widespread among Enterobacteriaceae. The present invention meets this requirement through its ease of use and its speed. The invention is based on detecting the enzyme activity of β-lactam hydrolysis using an antibody capable of discriminating between the intact form of the β-lactam ring of a β-lactam and its hydrolysis product. This antibody can be used in kits and methods that make it possible to rapidly detect (in less than one hour), without using expensive equipment (a small strip visible to the naked eye), the presence of bacteria producing penicillin-type, plasmid-mediated or hyper-produced AmpC enzymes, ESBL or carbapenemase from colonies or in a sample.

### DESCRIPTION OF THE PRIOR ART

Following the discovery of penicillin in 1928, its use has grown continuously, considerably reducing mortality linked to infectious diseases. However, as early as 1940, the first resistance to penicillin was identified, calling into question its effectiveness against certain germs (Maugat, Berger-Carbonne, et Agence nationale de sécurité sanitaire de l'alimentation, de l'environnement et du travail (ANSES), «Consommation d'antibiotiques et resistance aux antibiotiques en France: une infection évitée, c'est un antibiotique préservé!», 2018). In general, this phenomenon occurs a few years after the introduction of a new antibiotic, due to its massive and repeated use in human and animal health which, over time, generates an increase in resistance (Iredell, Brown, et Tagg, «Antibiotic Resistance in Enterobacteriaceae», 2016). In fact, antibiotics act not only on the bacteria responsible for the infection to be treated, but also on all the bacteria which constitute the various human, animal and environmental microbiota. Thus, all bacteria are capable of developing resistances to antibiotics, either by chromosomal mutations or by acquisition of new resistance mechanisms, in addition to those which some bacteria possess naturally (Maugat, Berger-Carbonne, et Agence nationale de sécurité sanitaire de l'alimentation, de l'environnement et du travail (ANSES), «Consommation d'antibiotiques et résistance aux antibiotiques en France: une infection évitée, c'est un antibiotique préservé!», 2018). The appearance and emergence of such resistance is not surprising, because the majority of antibiotics are derived directly or indirectly from natural microbial products (Iredell, Brown, et Tagg, «Antibiotic Resistance in Enterobacteriaceae », 2016).

At the start of the 1980s, the development of third generation cephalosporins (3GC) enabled enterobacteria to be combated effectively. However, as early as 1983, a first case of resistance was observed in Europe with the appearance of extended spectrum β-lactamase bacteria (hereinafter “ESBL” bacteria), other than the already known narrow-spectrum penicillinases and cephalosporinases. β-lactamases cause the amide bond of the β-lactam ring to open, making the antibiotic agent ineffective and the bacteria resistant to its deleterious effect. Bacteria producing an ESBL are capable of hydrolysing the β-lactam rings present in β-lactams such as penicillin, as well as the various classes of cephalosporins (1GC, 2GC, 3GC, 4GC, 5GC). They are mainly expressed by Gram-negative bacteria and more particularly in enterobacteria, which are the main sources of antibiotic resistance (Bonomo, «β-Lactamases», 2017). In the 1990s, new CTX-M type BSBLemerged and rapidly became the most widespread β-lactamases in enterobacteria clinical isolates. Currently they are mainly present in Escherichia coli and Klebsiella pneumoniae (Cattoir, «Les nouvelles β-lactamases à spectre étendu (BLSE)», 2008).

These ESBL-producing bacteria constitute a genuine public health problem. More specifically, these bacteria are more and more frequently isolated and require treatments, for serious infections, based on antibiotics of last resource (carbapenems), which is leading to the appearance of resistance to carbapenems with carbapenemases (Perez et al., «The Continuing Challenge of ESBLs»). This development of antibiotic resistance has led to a growing number of therapeutic dead-ends, thus causing infectious diseases, if nothing is done, to become one of the leading causes of mortality from 2050 onwards.

Current treatment of serious ESBL-producing Enterobacteria infections relies on carbapenems or on certain novel β-lactamase inhibitors (avibactam, relebactam, vaborbactam), because BSBLs do not have the ability to hydrolyse these molecules.

However, these antibiotics are used as a last resort, in order to limit the appearance of strains resistant to them. An antibiotic treatment based on cephalosporins is often prescribed as a first response, but it proves ineffective if the infecting bacteria produce an ESBL. Hence, it is necessary to adjust the antibiotic therapy to each type of infectious bacteria, according to its antibiogram, and to do this as soon as possible in order to have an optimum treatment and a high therapeutic success rate (Maugat, Berger-Carbonne, et Agence nationale de sécurité sanitaire de l'alimentation, de l'environnement et du travail (ANSES), «Consommation d'antibiotiques et résistance aux antibiotiques en France: une infection évitée, c'est un antibiotique préservé!»).

To this effect, it is essential to have powerful, simple, quick and transportable tools, for reliably identifying antibiotic-resistant bacteria, and in particular bacteria hydrolysing 3GC and which require an antibiotic treatment of last resort.

Various genotypic and phenotypic methods have been developed over the past few years for detecting and identifying bacteria possessing β-lactamase activity and therefore capable of resisting β-lactams. Each of these methods encompasses a multitude of tests which respond to different needs and objectives. There is no single test which is ideal in all situations, which explains the motivation for developing these diagnostic tests (Aguirre-Quiñonero et Martinez-Martinez, 2017).

In particular, the following methods have been described in patent documents:
WO2008/114001

This international patent application describes a kit for detecting ESBL bacteria, which consists of a medium containing an antibiotic for killing Gram positive bacteria, an anti-fungal compound, an antibiotic to kill off the non-ESBL Gram negative bacteria, and a coloured indicator. The presence of ESBL bacteria is detected by placing the sample to be tested on said support for 18-24 hours at 36-37° C. and by detecting the change in colour of the pH indicator, due to the acidification of the medium linked to the growth of the ESBL bacteria. This colorimetry method is simple to use but the interpretation of the colour change is subjective. Moreover, this colour change is induced by acidification of a medium buffered by bacterial growth. For this reason, a relatively large incubation time (16-24 hours) is required to reveal the presence of the sought bacteria.
WO2011/154517

The method described in this international patent application requires placing a bacterial suspension in contact with an appropriate substrate (a β-lactam antibiotic or a customised derivative of β-lactam) for several hours, then measuring with a mass spectrometer (MALDI-TOF) the appearance of peaks corresponding to the hydrolysed products of the antibiotic if the bacteria contain β-lactamases. In the presence of active β-lactamases, the molecular weight of the substrate is modified; the peak for the hydrolysed product appears while the peak for the intact substrate reduces. This method has disadvantages such as expensive equipment, qualified labour, a lack of software for automatic interpretation of the mass spectra (to determine the peaks and the exact masses expected), the non-adaptability in the field and a sometimes vague standardisation of the protocol (different incubation times, substrates used, calibration of the mass spectrometer, bacterial lysis conditions, etc.).
WO2011/107703

The detection method described in this application relies on the use of a chromogenic or fluorogenic substrate of β-lactamases enabling the presence of ESBL bacteria to be detected. In order to implement it, it is necessary to: a) concentrate the microorganisms present in the biological sample, optionally after a step of culturing microorganisms; b) suspend the microorganisms that were concentrated in step a) in a solution comprising at least one chromogenic or fluorogenic substrate of β-lactamase that is able to release a chromophore or a fluorophore after hydrolysis by the β-lactamase enzyme to be detected; c) detect the possible release of the chromophore or fluorophore obtained in step b), the detection of the release of the chromophore or fluorophore being indicative of the presence of β-lactamase and therefore of ESBL bacteria. The hydrolysis of the substrate causes the appearance of a coloured or fluorescent signal in the medium. However, the incubation time can be relatively long for enzymes having a weak enzymatic activity. Furthermore, some colourings which are not very pronounced can be difficult to interpret and a reading device is required for the fluorogenic substrates. Moreover, these substrates can be sensitive to light, which poses a problem if the test must be adapted in the field.
WO 2013/72494

The method described in this application aims to detect bacteria producing extended spectrum β-lactamases (extended spectrum β-lactamase hydrolysing cephalosporin). This method comprises the following steps: a) performing cellular lysis of the sample; b) reacting a fraction of the suspension obtained in step a) with a reagent kit comprising: i) a substrate of extended spectrum β-lactamase chosen from the group consisting of cephalosporins, aztreonam and cephamycins, and ii) a pH indicator which changes colour when the pH of the solution is between 6.4 and 8.4. This change of pH is induced by hydrolysis of the cephalosporin present in the medium, which causes the appearance of a carboxylic acid function. The change of colour in step b) indicates the presence of extended spectrum β-lactamase-producing bacteria in the sample. This calorimetry method is simple to use but, like WO2008/114001, the interpretation of the colour change is subjective. Moreover, this colour change is induced by acidification of a medium buffered by bacterial growth. For this reason, a relatively large incubation time (16-24 hours) is required to reveal the presence of the sought bacteria.
WO2016/156605

This electrochemical method can determine the presence of ESBL bacteria through their electrochemical properties, visible using an apparatus. This technique has a high usage cost (expensive equipment and qualified personnel).
US2018/156796

This American patent application describes methods for detecting the presence of bacteria that are resistant to antibiotics with a β-lactam ring in a sample, by placing the sample in contact with antibodies which specifically recognise molecules containing a hydrolysed β-lactam ring. The hydrolysed form of the antibiotic is immobilised on a support, for example on a strip. In these methods, the specific labelled antibody of the hydrolysed antibiotic is placed in contact with bacteria of the sample, then the mixture is deposited on the strip on which the hydrolysed antibiotic is immobilised. If the sample contains antibiotic-resistant bacteria (“positive” result), then the labelled antibody is saturated by the hydrolysed antibiotic present in the sample in large quantity, and will not therefore bind on the immobilised antibiotic of the strip: there is then no signal on the test zone of the strip. In the contrary case, i.e. if the sample contains non-ESBL bacteria (“negative” result), then the antibody introduced into the sample remains free to bind to the hydrolysed antibiotics immobilised on the strip, and the signal becomes strong on the test zone. On detecting the appearance of the product of the enzyme reaction that it is sought to detect (i.e., the hydrolysed form of the antibiotic), the presence of the active enzyme in the sample is thus manifest by a reduction in the signal.

When choosing a detection strategy, several factors must be taken into account, such as: the cost, the time required for obtaining results, the performance of the test and the information gathered by the test. The identification of the antibiotic resistance must be as quick and accurate as possible, in order to adjust the therapy of infected patients in the best possible way and as soon as possible, and to limit to a maximum the dissemination of resistant strains, by identifying infected or colonised patients. These tests are therefore very important for health professionals and for those involved in the prevention of bacterial infections on the local regional and national level (Lecour, «Détection des carbapenemases chez les entérobactéries»; Lutgring et Limbago, «The Problem of Carbapenemase-Producing-Carbapenem-Resistant-Enterobacteriaceae Detection», 2016).

The present invention meets this requirement through its ease of use and its speed. It is based on the detection of enzymatic β-lactam hydrolysis activity, using an antibody specifically recognising the intact form of the β-lactam ring of an antibiotic. This antibody is then used in immunochromatographic kits and tests for obtaining a very quick result (in less than one hour), without using expensive equipment (a strip that is readable with the naked eye). The use of such an antibody makes it possible to link the presence of active enzymes or ESBL bacteria in a sample (“positive” result) with the appearance of a signal, and not the reverse as proposed by many documents of the prior art. Indeed, through the antibody of the invention (recognising the antibiotic of the intact β-lactam ring), it is possible to measure the disappearance of the substrate (intact antibiotic which is introduced into the sample) in a reliable, specific and reproducible manner, by measuring the appearance of a signal (which is easier than observing the reduction of a signal).

For this reason, the test of the invention is extremely reliable: a sensitivity of 100% and a specificity of 100% have been obtained for the detection of cephalosporinase activity (enzymatic activity of cefotaxime hydrolysis) in many tested bacteria, in 40 minutes (cf. examples below).

The detection kits and methods that are the object of the present invention can:
facilitate the detection of bacteria capable of hydrolysing β-lactams (a coloured signal is synonymous with positivity)
optionally, identify what type of enzyme is expressed by the detected bacteria,
reduce the time for detecting bacteria capable of hydrolysing β-lactams (no longer than 40 minutes is now required to obtain a result),
detect all bacteria producing β-lactamases, whether known or unknown (a conclusion is possible even in the case of the appearance of new mutations)
use immunochromatography, which has many advantages in terms of rapidity, cost, simplicity and sensitivity.

### DESCRIPTION OF THE INVENTION

The present invention relates to novel means for detecting the hydrolysis of β-lactam, and thus for revealing the presence of β-lactamase-producing bacteria in a sample. These novel means are based on the use of an antibody specifically developed by the inventors, in order to recognise an antibiotic with intact β-lactam ring (and not its hydrolysis product). This antibody can advantageously be used in detection kits and detection methods described below. Very advantageously, these kits contain a strip (which is also an aspect of the invention itself), on which the antibody of the invention has been deposited and dried.

In the context of the present invention, monoclonal antibodies have been produced and selected for specifically recognising the intact form of β-lactam type antibiotics (i.e., comprising a β-lactam core) (cf. examples 1 and 2). In order to achieve this objective, immunogens (containing an intact β-lactam ring) and selection tests have been designed and implemented. Examples 1 and 2 of the application, presented below, describe how these immunogens have been designed and then produced, and how highly discriminating antibodies were then able to be selected. The antibiotics which were used in these examples are cefotaxime, a third generation cephalosporin (example 1), and meropenem, a new generation antibiotic which is hydrolysed by certain ESBL enzymes called “carbapenemases” (example 2).

As explained in detail in the examples below, these monoclonal antibodies were obtained by proceeding through the following steps:
A) coupling an antibiotic containing an intact β-lactam ring, with a large immunogenic molecule (BSA or haemocyanin type), far from the β-lactam core. This coupling was carried out by performing an acetyl chloride activation of the antibiotic, then grafting thiol functions onto the large molecule, then placing the two activated molecules in contact (this coupling step can also be carried out by various conventional chemical means that are known to a person skilled in the art).
B) immunising mice by injecting the animals with a sufficient quantity (for example 50 μg) of the intact antibiotic coupled with the large immunogenic molecule.
C) sampling the antibodies produced by the immunised animals, and identifying antibodies recognising the intact β-lactam ring (see the test presented in FIG. 1).
D) producing hybridomas producing the selected antibodies (from the splenocytes of immunised mice which have been fused with murine myeloma cells).
E) isolating each hybridoma produced in a well and confirming that the monoclonal antibodies produced recognise the antibiotic for which the β-lactam ring is intact (cf. test of FIG. 1 and test 1 of FIG. 9).
F) identifying and selecting hybridomas producing antibodies which do not recognise at all the antibiotic for which the β-lactam ring is hydrolysed (negative antibodies in test 2 of FIG. 2 and FIG. 9).
G) determining the specificity of each selected antibody with respect to the antibiotic with intact β-lactam ring (tests 3 and 4 of FIG. 2 and FIG. 9) with various concentrations of the intact and hydrolysed antibiotic).

By using this protocol, the inventors have produced many hybridomas producing different discriminating antibodies, having a very strong affinity for the intact form of the β-lactam ring (weaker signal in the presence of the inhibitor in test 3 of FIG. 2 and FIG. 9, no reduction in signal in the presence of the inhibitor in test 4 of FIG. 2 and FIG. 9). These antibodies are very specific for the intact form of the antibiotic (no cross-reactions observed with the hydrolysed form; cf. FIG. 3).

Other discriminating antibodies according to the invention can be generated by reproducing these steps, starting from another antibiotic.

### Antibody of the Invention

In a first aspect, the present invention thus relates to a monoclonal antibody specifically recognising an antibiotic molecule containing an intact β-lactam ring, this antibody not recognising the same antibiotic molecule when hydrolysed, i.e., when its β-lactam ring has been hydrolysed, for example by a β-lactamase. Hence, the antibody of the invention is capable of binding uniquely on antibiotics for which the β-lactam ring is intact. It can therefore discriminate between the two forms of the antibiotic, since it will be complexed when it is placed in contact with the intact form of the antibiotic, but will remain free in the presence of the hydrolysed form thereof. It is then sufficient to detect the existence of these complexes, in order to know whether the antibiotic was in the intact or hydrolysed form. This is why the monoclonal antibody of the invention will hereinafter be called a “discriminating antibody” according to the invention.

Within the meaning of the present invention, the term “intact” is synonymous with “non-hydrolysed”. Hence, an “intact” β-lactam ring is a β-lactam ring which is closed, because it has not undergone hydrolysis by a β-lactamase. By extension, an “intact” or “non-hydrolysed” antibiotic, is an antibiotic for which the β-lactam ring is closed and therefore functional (more specifically this ring provides the antimicrobial effect of the antibiotic). On the other hand, the term “hydrolysed” designates an antibiotic for which the β-lactam ring is open, because it has been naturally (in a natural sample) or artificially (for example by a synthesis enzyme) hydrolysed by a β-lactamase. A “hydrolysed” antibiotic is generally non-functional, i.e. it has little or no antimicrobial effect.

As used here, the term “monoclonal antibody” refers to an antibody from a homogeneous population of antibodies. More particularly, the individual antibodies of a population of monoclonal antibodies are identical. In other words, a monoclonal antibody consists of a homogeneous population of antibodies originating from the growth of a monocellular clone (for example a hybridoma, a host eukaryote cell transfected with a DNA molecule coding for the homogeneous antibody, a host prokaryote cell transfected with the DNA molecule coding for the antibody, etc.). It is generally characterised by heavy chains and light chains. Monoclonal antibodies are highly specific and are directed against a single antigen. An “antigen” is a predetermined molecule on which an antibody can bind selectively to a region referred to as an epitope. In the context of the invention, the target epitope includes the β-lactam ring of an antibiotic.

Here, the term “specifically recognising” shall mean that the monoclonal antibody of the invention has a very strong affinity for the intact target antibiotic and a very weak infinity for the antibiotic which has been hydrolysed, for example by a β-lactamase. Preferably, its dissociation constant K_{d} for the target antibiotic is between approximately 10 nM and approximately 1 pM. More preferably, said K_{d} is between approximately 10 pM and approximately 40 pM. The expression “K_{d}” refers to the dissociation constant of a given antibody-antigen complex. K_{d}=k_{off}/kₒₙ with k_{off} consisting of the “off rate” constant for the dissociation of the antibody of the antibody-antigen complex and kₒₙ being the level at which the antibody combines with the antigen (Chen Y. et al., 1999, J. Mol. Biol., 293:865-881). It is also possible to measure the affinity of the antibody of the invention with its target by measuring its association constant, Kₐ, which corresponds to the inverse of K_{d}. Preferably, the association constant Kₐ of the antibody of the invention for the target antibiotic (having an intact β-lactam ring) is greater than approximately 10⁹ M⁻¹, more preferably greater than 10¹¹ M⁻¹ and still more preferably, greater than 10¹² M⁻¹.

Antibodies having a weak affinity for a target generally bond slowly to this target and have a tendency to dissociate easily, whereas antibodies with high affinity for a target generally bond to the target rapidly and have a tendency to remain bonded to it for a longer time. A variety of methods for measuring bond affinity are known in the art (for example by dialysis at equilibrium, or by fluorescence, or else with Biacore analyses), any of which can be used for the purposes of the present invention. It is also possible to use, for example, the tests shown in FIGS. 2 and 9.

The present invention also targets those fragments of monoclonal antibodies which are functional (i.e., which specifically recognise an antibiotic molecule for which the β-lactam ring is intact, but not when it is hydrolysed). This fragment can be, for example, chosen from the fragments Fv, Fab, (Fab′)₂, Fab′, scFv, scFv-Fc and the diabodies.

The monoclonal antibody of the invention can be produced and isolated by conventional means using any known technique enabling the production of antibody molecules by culture cell lines. The techniques for producing monoclonal antibodies include, but are not limited to, hybridoma techniques, human B-cell hybridoma technique and the EBV-hybridoma technique.

The examples of the application describe how to obtain antibodies according to the invention, specifically recognising cefotaxime or meropenem as target antibiotic. Other target antibiotics can be used.

Within the meaning of the invention, the term “target antibiotic” designates any antibiotic known to contain, in its chemical formula, a β-lactam ring. Otherwise known as “beta-lactam antibiotic” or “β-lactam antibiotic”, it can be chosen from the penicillins, cephalosporins, monobactams, and carbapenems, which all contain a β-lactam core in their molecular structure.

The target antibiotic can be, in particular, a penicillin chosen from benzylpenicillin (penicillin G), phenoxymethylpenicillin (penicillin V), meticillin, dicloxacillin, fucloxacillin, amoxyicillin, ampicillin, piperacillin, ticarcillin, azlocillin, and carbenicillin.

The target antibiotic can be, in particular a cephalosporin chosen from cephalexin, cephalotin, cephazolin, cefaclor, the cefuroxim, cefamandole, cefotetan, cefoxitin, ceftriaxone, cefixin, cefotaxime, ceftazidime, cefepime, cefpirome, ceftaroline and ceftobiprole.

The target antibiotic can be, in particular, a carbapenem chosen from thienamycin, imipenem, meropenem, ertapenem, biapenem, tebipenem and doripenem. These new-generation antibiotics are not hydrolysed by all the ESBL, but only by certain ESBL enzymes called “carbapenemases”. It can be advantageous to immunise animals with this type of molecule, in order to generate antibodies discriminating between their intact form and their hydrolysed form, and able to detect the presence of carbapenemase enzymes in a sample.

The target antibiotic can be, in particular, a monobactam such as aztreonam.

The target antibiotic can be, in particular, a cephamycin chosen from cefmetazole and latamoxef.

In a preferred embodiment, the discriminating antibody of the invention is characterised in that it specifically recognises an antibiotic molecule chosen from penicillins, cephalosporins, monobactams, carbapenems and cephamycins, for which the β-lactam ring is intact.

In a more preferred embodiment, the antibody of the invention is characterised in that it specifically recognises an antibiotic molecule chosen from the group consisting of: benzylpenicillin (penicillin G), phenoxymethylpenicillin (penicillin V), meticillin, dicloxacillin, fucloxacillin, amoxyicillin, ampicillin, piperacillin, ticarcillin, azlocillin, carbenicillin, cephalexin, cephalotin, cephazolin, cefaclor, cefuroxim, cefamandole, cefotetan, cefoxitin, ceftriaxone, cefixin, cefotaxime, ceftazidime, cefepime, cefpirome, ceftaroline, ceftobirpole, thienamycin, imipenem, meropenem, ertapenem, biapenem, tebipenem, doripenem, aztreonam, cefmetazole and latamoxef, for which the β-lactam ring is intact.

In another more preferred embodiment, the antibody of the invention is characterised in that it specifically recognises an antibiotic molecule chosen from the group consisting of thienamycin, imipenem, meropenem, ertapenem and doripenem.

By contrast, said antibodies do not recognise (or have a poor affinity for) these antibiotics molecules when the β-lactam ring has been hydrolysed, for example by the effect of a β-lactamase enzyme.

In the context of the invention, the term “P-lactamase” refers to the following enzymes: penicillinases (e.g. TEM-1, SHV-1 . . . ), cephalosporinases (e.g. AmpC), extended spectrum β-lactamases (e.g. derivatives of TEM, SHV, CTX-M . . . ), as well as carbapenemases.

Here, “extended spectrum β-lactamase enzyme” or “ESBL” shall mean an enzyme having a spectrum of penicillinase having broadened its spectrum with respect to third-generation cephalosporins, and remaining inhibited by clavulanic acid.

The “extended spectrum β-lactamases” (or “ESBL”) are a large and very heterogeneous family of bacterial enzymes discovered in the 1980s in France and then in Germany. They are induced either by plasmids (frequent), or by mutation of the natural genome in Klebsiella spp, coding for an SHV β-lactamase. The two mechanisms give the affected bacteria the capacity to hydrolyse a large variety of penicillins and cephalosporins. The majority of ESBL are the result of genetic mutations of natural β-lactamases, in particular of TEM-1, TEM-2 and SHV-1. They are very active against penicillins and moderately active against first-generation cephalosporins. The genetic mutations at the origin of ESBL extend the spectrum of these enzymes and also affect third-generation cephalosporins (ceftazidime and cefotaxime) and monobactams (aztreonam).

The examples below explain how to identify the existence of antibodies according to the invention, by using an antibiotic molecule (here cefotaxime or meropenem) that is intact or hydrolysed by means of an enzyme hydrolysing the antibiotic concerned (here the CTXM-2 enzyme or carbapenemase of Klebsiella pneumoniae).

The affinity of the antibody of the invention for the hydrolysed form of the antibiotics is very low. It is, for example, such that either the dissociation constant K_{d} is much greater than that for the intact antibiotic (for example 100 times greater than) or its association constant Kₐ is very much less than that for the intact antibiotic (for example 100 times less).

The examples of the application demonstrate how to obtain antibodies specifically recognising the intact target antibiotic, cefotaxime or meropenem, These antibodies do not recognise cefotaxime/meropenem when it has been hydrolysed with a β-lactamase enzyme. The tests shown in FIG. 2 and FIG. 9 make it possible to select the appropriate antibodies which can be used to detect the ESBL bacteria in the methods of the invention.

In certain embodiments presented below, it can be advantageous to have the antibody of the invention available in detectable form. This is why the antibody of the invention is preferably detectably labelled, for example it is coupled to a fluorochrome, a radioactive ion, a contrast agent, a metal ion, to a chromophore, an enzyme or any other marker visible to the naked eye or detectable by imaging.

### Use of the Antibody of the Invention for Detecting a β-Lactamase Hydrolysing a 3GC

The invention also relates to the use of a discriminating antibody according to the invention (as described above) in tests for rapidly and very reliably detecting, in any sample, the enzymatic activity of a β-lactamase enzyme and therefore the presence of bacteria having a β-lactamase activity (otherwise known as “β-lactam-resistant antibiotics). More precisely, the antibody of the invention can be used to rapidly and reliably detect the presence of bacteria having an extended spectrum β-lactamase (ESBL) activity in a sample. The use of an antibody for detecting the presence of such bacteria has never been proposed in the art.

In a second aspect, the present invention therefore covers the use of the discriminating antibody of the invention, as described above, for detecting the presence of a functional, preferably extended spectrum, β-lactamase enzyme, in a sample.

In the context of the invention, the term “sample” means any solid or liquid, biological or environmental fraction, that is capable of containing a β-lactamase enzyme as described above, or bacteria expressing such an enzyme, said enzyme being capable of being functional. It may involve, for example, an environmental sample, or else a human, animal or plant biological fluid.

Preferably, the sample used in the methods of the invention contains bacteria.

As used here, the term “biological fluid” refers to any sample that has been obtained from an individual human, animal, or plant, and which is fluid or viscous. It may, for example, be a biological liquid produced by a human or an animal, such as urine, cerebrospinal fluid, pleural fluid, synovial fluid, peritoneal fluid, amniotic fluid, gastric fluid, blood, serum, plasma, lymphatic fluid, interstitial fluid, saliva, physiological secretions, tears, mucus, sweat, milk, sperm, seminal fluid, vaginal secretions, a fluid from ulcers and other surface eruptions, blisters, faeces or abscesses. It can alternatively be a fluid generated by (or which has been in contact with) a plant such as sap, run-off water or dew.

In a preferred embodiment, said sample, whatever it is, contains bacteria.

The antibody of the invention can be used in any immunological test that enables a reduction in the quantity of intact antibiotic to be detected unequivocally when it is placed in contact with the sample. This reduction being due to the action of β-lactamase enzymes present in the sample, the antibody of the invention will thus be able to reveal their presence.

The antibody of the invention is preferably used in a biological test by competition, i.e. in a test in which the presence of a β-lactamase will be revealed by the appearance (and not the reduction) of a detectable signal. In such a test by competition, the antibody of the invention will be either labelled or immobilised, and will be placed in contact with the antibiotic with intact ring which it specifically recognises, which will itself be labelled or immobilised. In a preferred embodiment, said test involves a labelled (but mobile) antibody of the invention, and an immobilised intact antibiotic. In another preferred embodiment, said test involves the immobilised antibody of the invention, and the unlabelled and labelled (but mobile) intact antibiotic. In another preferred embodiment, said test is carried out entirely in the liquid phase, and involves using the antibody of the invention and associated antibiotics which will have been labelled by means of various detectable markers. If different fluorophores are used, it will be possible to measure the co-location of the antibodies/antibiotics using the FRET technique.

It is for example possible to detect the presence of activated β-lactamase by comparing the signal obtained under the following conditions:
when the sample is incubated in the presence of the intact antibiotic before being placed in contact with immobilised antibody of the invention in wells or on a support, in the presence of a labelled intact antibiotic (with biotin or with any other marker);
when the antibody of the invention is placed in contact with the intact antibiotic without having been placed in contact with the sample or having been placed in contact with a sample not containing an enzyme hydrolysing the antibiotic, before being placed in contact with the labelled intact antibiotic (the signal is then minimum).
Alternatively, the antibody of the invention is placed in contact with the intact antibiotic after its incubation with a purified β-lactamase, before being placed in contact with the labelled intact antibiotic (the signal is then maximum).

Of course, in order to perform such experiments, it is necessary that the antibiotic used (in intact form) is specifically recognised by the antibody of the invention.

It may be useful, in certain preferred embodiments, to also use antibodies which can detect certain CTX-M enzymes or carbapenemases, so as to characterise which type of enzyme is responsible for the ESBL detected using the antibodies and methods described in the present invention. It will be possible to carry out this detection of the activity and identification of the enzyme involved simultaneously during a given test.

### Methods and Kits of the Invention

In a particular aspect, the invention relates to methods for detecting bacteria producing functional β-lactamase enzymes, using antibodies according to the invention and the antibiotics that they specifically recognise.

When an antibiotic having a β-lactam ring, as described above, that is in intact form and optionally labelled, is available, together with an optionally labelled antibody for specifically detecting it (because it is produced as described above using said antibiotic as an immunogenic agent), said method can advantageously comprise the following steps, in this order:
a) placing the sample to be tested in contact with said intact antibiotic,
b) adding said antibody to the sample,
c) detecting whether said antibodies are complexed with the intact antibiotic.

It is then possible to compare the signal obtained with that which is generated when the antibodies and the intact antibiotic have not been placed in contact with the sample (when they were placed in contact with a sample not containing an enzyme hydrolysing the antibiotic), or when the intact antibiotic has been hydrolysed by a β-lactamase before being placed in contact with the antibodies.

It is, more precisely, possible to detect the presence of β-lactamase activity by comparing the signal obtained by using an immobilised antibody, an intact antibiotic and a labelled antibiotic, under the following conditions:
when the sample containing the enzyme hydrolysing the antibiotic is incubated in the presence of the intact antibiotic, before being placed in contact with immobilised antibody of the invention (in wells or on a support), prior to the addition of the labelled antibiotic (the signal is then maximum);
when the intact antibody is placed in contact with the antibody of the invention without having been placed in contact with the sample or is placed in contact with a sample not containing an enzyme hydrolysing the antibiotic, before the addition of the labelled antibiotic (the signal is minimum).

These methods have been designed in order that the signal detected in step d) is proportional to the quantity of β-lactamase enzymes present in the sample. The higher the concentration of the enzyme in the tested sample, the faster will be the hydrolysis of the intact antibiotic, and the more the antibodies added to the sample will remain free to complex with the intact antibiotic which will be subsequently placed in contact. On later detecting the binding of the intact antibiotic on the antibody of the invention, the intensity of the signal will be more intense the higher the concentration of enzyme initially present in the sample. These methods are very advantageous, since it is much easier and more reliable to detect the appearance of a signal than its reduction.

In a particular embodiment, the invention targets a method for detecting the presence of bacteria producing a functional, preferably extended spectrum, β-lactamase enzyme, said method using at least one antibody as defined in the invention and the antibiotic molecule containing an intact β-lactam ring specifically recognised by said antibodies.

More specifically, said method uses i) an antibiotic having an intact β-lactam ring, as described above, in a labelled form and/or in an unlabelled form, and ii) at least one antibody specifically making it possible to detect this intact antibiotic, produced as described above by using said antibiotic or an intact analogue such as an immunogenic agent, said antibodies being immobilised on a solid support or easily detectable.

The method can also be used, in addition to specific intact antibodies, for known capture antibodies for recognising the hydrolytic enzymes expressed by bacteria. This step makes it possible, if the bacteria of the sample have an ESBL activity, to determine which enzyme is responsible for this activity and to classify the bacteria as a function of this parameter (or detect an activity due to an unknown type of enzyme). It is possible, in particular, in order to complete the information according to which the studied sample contains ESBL bacteria, to use anti-CTX-M or anti-carbapenemases antibodies known in the art (for example those described in Bernabeu et al., 2020; Boutal et al., 2018).

The present invention also relates to a kit for implementing such methods. This kit contains at least the antibody of the invention and possibly the antibiotic which was used to produce it. It further contains, optionally, means for labelling the antibiotic and/or the antibodies in order to be able to detect it or them. In a particular kit, the antibody and/or the antibiotic is/are immobilised on a solid support (for example a strip), or are already labelled.

A method according to the invention comprises, for example, the following steps, in this order:
a) placing the sample to be tested in contact with said antibiotic containing an unlabelled intact β-lactam ring,
b) placing the sample in contact with said antibody which has been immobilised on a solid support (or which is detectable),
c) placing the antibiotic containing a labelled intact β-lactam ring, in contact with said antibody, or with the sample of step b) containing said antibody,
d) detecting whether said antibody is complexed with the labelled intact antibiotic placed in contact in step c),

If β-lactamase is not present in the sample, the (unlabelled) antibiotic added to the sample (step a) remains in intact form, the antibodies of the invention will bond to this intact antibiotic and will not be able to bind the intact antibiotic labelled in step c). By contrast, if the enzyme is present in the sample, the (unlabelled) antibiotic will be hydrolysed in step a), and the antibody of the invention will remain free to bind on the intact antibiotic labelled in step c). The signal detected in step d), when the labelled antibiotic binds on the antibody of the invention, is thus proportional to the presence of enzyme in the sample (the signal increases the more enzyme is in the sample, since the imposed competition is to the detriment of the labelled antibiotic, which is added after the unlabelled antibiotic).

It is then possible to advantageously compare the signal obtained in the presence of the sample with that which is generated when the antibody and the intact antibiotic have not been placed in contact with the sample (or with a sample not containing an enzyme hydrolysing the antibiotic), or when the intact antibiotic has been hydrolysed by a β-lactamase before being placed in contact with the antibody (the negative and positive controls).

It is also possible, instead of using labelled and unlabelled antibiotics, to use antibiotics labelled in different ways, and that can be distinguished from one another.

If the antibody is immobilised, the appearance of the signal can be observed where the antibody is immobilised. If the antibody is labelled in a detectable manner, it is possible to observe the co-location of the two antibody/antibiotic labels, for example by fluorescence resonance energy transfer (FRET).

In the case where the user wishes to identify which β-lactamase or ESBL enzymes are expressed by the bacteria and prove to be active due to the tests of the invention, said user can also place the sample containing the bacteria in contact with known antibodies specifically recognising these enzymes (for example anti-CTX-Ms or carbapenemases), these antibodies having been labelled and/or having been immobilised for better subsequent detection.

The present invention also relates to a kit for implementing such methods. This kit contains at least the antibody of the invention and optionally the antibiotic which was used to produce it. It also contains, optionally, means for labelling the antibiotic so as to be able to detect it and/or known antibodies for detecting β-lactamase enzymes. In a particular kit, the antibody is supplied already immobilised on a solid support (for example a strip), or already labelled.

In another particular embodiment, the invention relates to a method for detecting bacteria producing functional β-lactamases, said method using at least i) an antibiotic having a β-lactam ring, as described above, in intact form, and ii) an antibody for specifically detecting this intact antibiotic, produced as described above by using said antibiotic as an immunogenic agent, said antibodies being labelled so as to be detectable.

A method according to the invention comprises, for example, the following steps, in this order:
a) placing the sample to be tested in contact with the antibiotic containing an intact β-lactam
b) placing the sample after step a) in contact with the antibody, which has been labelled,
c) placing the antibody obtained after step b) in contact with the antibiotic containing an intact β-lactam ring, which has been immobilised on a solid support, or adding the intact antibiotic that is labelled separately from the antibody,
d) detecting whether said labelled antibody is complexed with the intact antibiotic placed in contact in step c).

If β-lactamase is not present in the sample, the antibiotic (unlabelled after step a) added to the sample will remain in intact form, the antibodies of the invention will bond to this intact antibiotic and will not be able to bind the intact antibiotic labelled in step c). By contrast, if the enzyme is present in the sample, the (unlabelled) antibiotic added in step a) will be hydrolysed, and the labelled antibody of the invention in step b) will remain free to bind on the immobilised or labelled intact antibiotic, during step c). The signal detected in step d), when the immobilised antibiotic binds on the labelled antibody of the invention is thus proportional to the presence of enzyme in the sample (the signal increases more the more enzymes are in the sample, since the imposed competition is to the detriment of the immobilised antibiotic).

As previously, it is possible to advantageously compare the signal obtained in the presence of the sample with that which is generated when the antibodies and the intact antibiotic have not been placed in contact with the sample (or with a sample not containing an enzyme hydrolysing the antibiotic), or when the intact antibiotic has been hydrolysed by a β-lactamase enzyme before being placed in contact with the antibodies (the negative and positive controls).

If the antibiotic is immobilised, the appearance of the signal can be observed where the antibiotic is immobilised. If the antibiotic is labelled, it is possible to observe the co-location with the antibody of the invention, for example by fluorescence resonance energy transfer (FRET).

As mentioned above, in the case where the user wishes to identify which are the β-lactamase or ESBL enzymes, he can also place the sample containing the bacteria in contact with known antibodies specifically recognising these enzymes (for example anti-CTX-Ms or carbapenemases), these antibodies having been labelled and/or having been immobilised for better subsequent detection.

In this particular embodiment, the method of the invention comprises for example the following steps:
a) placing the sample to be tested in contact with the antibiotic molecule containing an unlabelled intact β-lactam ring,
b) placing the sample obtained after step a) in contact with the antibody of the invention (which specifically recognises the intact form of the antibiotic added in step a) and with at least one antibody specifically recognising a β-lactamase enzyme (for example CTX-M or carbapenemase), said antibodies having been labelled beforehand,
c) placing the solution of step b) in contact with said antibiotic molecule containing an intact β-lactam ring and with antibodies specifically recognising a β-lactamase enzyme (for example CTX-M or carbapenemase), which have been immobilised on a solid support,
d) detecting whether said labelled antibodies are complexed with the intact antibiotic and/or with the anti-β-lactamase antibodies complexed with the anti-β-lactamase enzyme, placed in contact in step c).

The present invention also relates to a kit for implementing such methods. This kit contains at least the antibody of the invention and optionally the antibiotic which was used to produce it. It also contains, optionally, means for labelling the antibody so as to be able to detect it and/or known antibodies for detecting β-lactamase enzymes. In a particular kit, the antibiotic is supplied in free form (for step a)) and also in immobilised form on a solid support (for example a strip), or already labelled.

Other more or less complex tests can be developed by using the discriminating antibody of the invention, as well as the antibiotic used to obtain it.

All the kits of the invention can also contain a β-lactamase enzyme which will be able to be used to verify the specificity of the antibody of the invention.

All the kits of the invention can also contain a control sample, containing no β-lactamase.

All the kits of the invention can also contain means for detecting the labelled antibody of the invention (for example, antibodies recognising the constant portion of a mouse immunoglobulin).

Finally, all the kits of the invention can contain instructions for explaining to users the details of the experiments to be carried out in order to detect bacteria producing functional β-lactamases in a quick and effective manner.

### Strip of the Invention

In a particular embodiment, the immunological test of the invention is an immunochromatographic test having as support a detection “strip”. In this case, the test of the invention is called a “strip test” and the solid support used in the method of the invention is a strip. This strip constitutes a particularly important aspect of the present invention.

Indeed, the present inventors have shown that the antibody of the invention can advantageously be used in a strip test. The antibody of the invention is thus preferably coupled to a fluorochrome or chromophore, or to any other marker visible to the naked eye, for example colloidal gold.

The “strip test” is a simple, fast and inexpensive detection system, which can be used by non-specialists, in the field. The strips are generally formed with three distinct zones, which are fixed on a support, that is usually plastic: 1) an absorption zone promoting migration, 2) a reaction zone (generally formed as a nitrocellulose membrane) and 3) a deposition zone where the sample to be tested is deposited, located at the opposite end from the absorption zone (cf. FIG. 5). An antibody, referred to as a “tracer” antibody because it is directed against the target to be detected and is bound to molecules enabling an, in general colorimetry or fluorescent, signal to be obtained, is deposited on the absorption zone and dried. In the context of the present invention, said “tracer” antibody will be the discriminating antibody of the invention, described above.

Two lines are generally present in the reaction zone. The first of these lines, termed the “test” line (TL), will be composed here of antibiotics with a β-lactam ring. The signal obtain at this test line will indicate the presence or otherwise of β-lactamase in the sample. A second line, referred to as the “control” line (CL), will consist of antibodies directed against the antibody of the invention.

Advantageously, antibodies recognising β-lactamase enzymes can also be immobilised on the strip of the invention, as proposed in FIG. 10. Preferably, these antibodies are labelled and deposited on the deposition zone and are also immobilised on one or more test lines but can be distinguished from that containing the immobilised antibiotic.

In this case, the strip according to the invention may contain a plurality of test lines and one control line. One test line will correspond to a zone where the intact antibiotic coupled to the BSA or to another carrying structure (for example casein, dextran or polylysine), has been immobilised, and the other test lines will correspond to zones where the anti-β-lactamase antibodies have been immobilised. In this case, the deposition zone will advantageously contain the antibody of the invention as well as the anti-β-lactamase antibodies, all labelled in a detectable manner (the markers used being distinct or identical).

In this particular embodiment, it is preferable to deposit, on the deposition zone, anti-β-lactamase antibodies recognising epitopes of the target enzyme which are different from those recognised by the anti-β-lactamase antibodies immobilised on the dedicated test line (the two populations of antibodies therefore recognise the same β-lactamase enzyme). Hence, the detection of the presence of the target enzyme will be even more reliable.

If the bacteria of the sample express a β-lactamase enzyme recognised by the labelled antibodies present on the deposition zone, these will be bound to the β-lactamase enzyme, and the enzyme will also be able to be bound on the anti-β-lactamase antibodies immobilised on the test zone. A labelling will therefore be visible on the test line corresponding to these antibodies, and it will be possible to deduce whether the bacteria bearing the ESBL activity express the enzyme recognised by the antibodies immobilised on this line.

The reaction zone can also contain a plurality of test lines on which various antibiotics are immobilised. In this case, a plurality of monoclonal antibodies, specific tracers of one of these antibiotics, will advantageously be present in the deposition zone. For example, the reaction zone can contain a test line on which an antibiotic A with intact β-lactam ring is bound, and a test line on which an antibiotic B with intact β-lactam ring, B, being different from A, is bound. In the deposition zone, monoclonal antibodies discriminating between intact antibiotics A and B and hydrolysed antibiotics A and B have been advantageously deposited, so that the signal would appear on two lines if there were β-lactamases in the tested sample.

In this context, it is also advantageous to deposit antibodies discriminating between the intact and hydrolysed forms of carbapenems, these new generation antibiotics which are known to be resistant to the ESBL enzymes other than carbapenemases. Example 2 below describes precisely how to obtain such antibodies, which can be used in the methods and kits of the invention.

In this particular embodiment, the antibodies discriminant between the intact and hydrolysed forms of a carbapenem are deposited on the deposition zone, and an intact carbapenem antibiotic is immobilised on a test line. The test of the invention can identify the presence, in the sample, of carbapenemase enzymes, and therefore potentially of bacteria expressing these enzymes.

In a still more preferred embodiment, the strip of the invention contains at least two test lines, on which are respectively immobilised the intact form of a non-carbapenem antibiotic (for example a cephalosporin) and the intact form of a carbapenem antibiotic, so as to identify in the sample the presence of ESBL enzymes other than carbapenemases (for example cephalosporinases, etc.) and carbapenemases. The respective discriminating antibodies that are labelled (possibly with different markers), are themselves deposited on the deposition zone. The strip of the invention can further contain, in its reaction zone, as explained above, other test lines, on which the anti-β-lactamase enzyme antibodies have been immobilised.

If the enzyme is not present in the sample, the antibiotic added to the sample will remain in intact form, the antibody of the invention will bind to this intact antibiotic and will no longer be able to bind on the intact antibiotic of the TL. By contrast, if the enzyme is present in the sample, the antibiotic added will be hydrolysed, and the antibody of the invention will remain free to bind on the immobilised intact antibiotic immobilised on the TL. In these two cases, the free antibodies, i.e. those which have not been immobilised on the test line, will be captured at the control line by the antibody of the invention.

The test strip of the invention has been designed in order that the signal at the test line increases with the quantity of β-lactamases present in the sample. The more concentrated the enzyme is in the tested sample, the faster will be the hydrolysis of the antibiotic, the less the antibody will complex with the added antibiotic, and the more it will bind on the TL: the signal intensity on the TL will therefore increase with this concentration.

Furthermore, the test strip of the invention is very reliable, in that it links the appearance of a signal (and not its disappearance) with the quantity of enzyme. However, it is well-known that it is easier and more reliable to detect the appearance of a signal than its reduction.

In this preferred embodiment, a strip according to the invention can be prepared in the following way:
the, preferably labelled, antibody of the invention is deposited, and for example dried, on the deposition zone,
Optionally, anti-β-lactamase antibodies, preferably labelled, are also deposited on the deposition zone.
The antibiotic with intact β-lactam ring is immobilised on the TL, in the reaction zone (for example with a BSA-antibiotic system, which enables the immobilisation of the antibiotic on the nitrocellulose while allowing it to interact with the labelled antibody).
Optionally, anti-β-lactamase antibodies are also immobilised on another line of the reaction zone,
antibodies recognising the antibodies deposited on the deposition zone of the strip (antibody of the invention and optionally anti-β-lactamase antibodies) are immobilised, for example by adsorption, on the CL, opposite the TL.

In a particular aspect, the present invention therefore relates to a strip containing (cf. FIGS. 5 and 10):
1) a zone for depositing the sample, on which the antibody as defined above has been labelled, deposited and dried, as well as, optionally, labelled anti-β-lactamase antibodies,
2) a reaction zone, comprising:
   at least one test line on which the antibiotic with intact β-lactam ring, having been used to produce at least one of the antibodies deposited in the deposition zone 1), has been immobilised, and
   optionally at least one test line on which anti-β-lactamase antibodies have been immobilised,
   a control line on which antibodies recognising the antibody or antibodies present on the zone 1) have been immobilised, and
3) an absorption zone promoting the migration of antibodies, said zone being situated at the end opposite the deposition zone 1).

The labelled antibodies are deposited and stored on the surface of the strip, preferably by drying. They can also be added to the sample just before depositing thereof on the strip.

The reaction zone is preferably made of nitrocellulose or PVDF, cellulose or glass fibre.

The immobilisation of the antibiotic on the TL is preferably achieved by adsorption of the antibiotic coupled to BSA or to another carrier molecule (for example casein, dextran or polylysine), which makes it possible to leave accessible the β-lactam ring that must capture the antibody of the invention migrating towards the absorption zone. It is also possible to absorb BSA-streptavidin complexed with the antibiotic coupled to biotin or to couple the antibiotic to beads with a diameter greater than the porosity of the membranes used for the reaction zone. Other binding techniques known in the art for immobilising small molecules can be used.

On the control line, the antibodies recognising the antibodies used on the strip of the invention are, for example, anti-murine-immunoglobulin-constant-portion antibodies, of protein A, protein G or any other system recognising murine antibodies.

As with conventional competition tests, the quantities of immobilised or labelled antibodies and antibiotics, or as substrate must be rigorously controlled. Too large a quantity of antibodies would require a large concentration of substrate in order for all the bond sites to be occupied and therefore a higher concentration of β-lactamase in order to have a significant reduction in the occupation of these sites. Similarly, too high a concentration of labelled or immobilised antibiotics would induce very high competition with respect to the bonding to the substrate by the antibodies, which would induce the appearance of a signal even in the absence of β-lactamase or else the use of an excess of substrate in order to maintain the total occupation of the bond sites of the antibody by this same substrate. This excess of substrate would lead to a strong reduction in sensitivity or to a longer incubation period.

Examples illustrating these optimisation steps are given below. It can be seen in table 5 that the concentration of substrate enabling a total disappearance of the signal is not identical depending on the quantities of labelled or immobilised antibodies and antibiotics, optimised for each of the antibodies. Hence, the concentrations of substrate for the antibodies 2 enabling the occupation of all the bond sites (which is manifest by the disappearance of the signal on the strip) are higher.

A person skilled in the art, monitoring the indications mentioned in the present invention, understands that it is important to adjust these parameters according to the chromophores and analytes used. Said person is capable, using current techniques and the explanations provided in the examples below, of identifying the optimum quantities of each analyte for an antibody having a given affinity for the intact antibiotic that it recognises. He understands, in particular, that the quantity of antibodies to be deposited on the deposition zone must be such that it can saturate all the sites for binding to the intact antibiotic used in the method or on the strip of the invention. Moreover, he understands that the minimum quantity is that which makes it possible to obtain a maximum signal on the test line containing the antibiotic.

For cefotaxime labelled with colloidal gold (example 1), the optimum quantity of antibody to be deposited on the deposition zone is such that 10 μL of the solution having an absorbance relative to the colloidal gold—DO—between 0.1 and 10 is deposited. Moreover, the optimum quantity of antibiotic immobilised on the line TL (1 μL/cm) is ideally between 1 μg and 1 mg/mL. When a coupling by BSA-antibiotic is used, a concentration of approximately 0.1 mg/mL gives, for example, excellent results.

In general, for other antibodies having a strong affinity for the intact antibiotic that they specifically recognise (K_{d} between 1 pM and approximately 10 nM), it is advantageous to immobilise, on the TL line of the strip, a quantity of intact antibiotic, by depositing 1 μL of solution per cm having concentrations between 10 μg and 1 mg/mL, between 50 μg and 1 mg/mL, between 100 μg and 1 mg/mL, or between 50 μg and 0.5 mg/mL and depositing on the deposition zone a quantity of antibody by using 10 μL of solution having a DO (if it is labelled with colloidal gold) between 0.1 and 10.

In order to facilitate the use of the strips of the invention, it is possible to insert the strips of the invention in plastic cassettes.

### Kits of the Invention, Containing the Strip of the Invention

Before being deposited on the deposition zone, an antibiotic with intact β-lactam ring must be added to the sample to be tested, prior to placing it in contact with the strip of the invention.

In a third aspect, the present invention also relates to a kit containing, in addition to the strip of the invention, the antibiotic with intact β-lactam ring which has been used to obtain the antibody of the invention, and which is immobilised on the deposition zone 1) of the strip. This antibiotic is preferably contained in a separate container, isolated from the strip.

For example, if the antibody of the invention has been produced by immunising animals with the antibiotic cefotaxime (cf. example 1 below), then the kit of the invention will contain a strip on which said intact anti-cefotaxime antibody is deposited and said intact antibiotic is immobilised, as well as a vial or a tube containing the intact cefotaxime antibiotic.

For example, if the antibody of the invention has been produced by immunising animals with the analogue carbapenem (cf. example 2 below), then the kit of the invention will contain a strip on which said intact anti-carbapenem antibody is deposited and said intact antibiotic is immobilised, as well as a vial or a tube containing the intact carbapenem antibiotic.

The user will thus have all the elements for carrying out the test of the invention and can extemporaneously add, to the sample to be tested, the reagent (antibiotic) which will make it possible to implement the test of the invention (cf. below).

In order to check that the test strip is properly specific, the kit of the invention can also include a container comprising a functional β-lactamase. The user can thus, for example, compare the difference in signal obtained in the sample to be tested (i.e., when the endogenous β-lactamase is potentially present), or when an exogenous enzyme is added. This step can be used to check that the test is properly functional.

As indicated above, this kit can also contain a control sample which does not contain β-lactamase, the means for detecting the labelled antibody of the invention (for example, antibodies recognising the constant part of a mouse immunoglobulin) and/or instructions for explaining to users the details of the experiments to be carried out in order to quickly and efficiently detect bacteria producing functional β-lactamases.

Hence, the present invention relates, more particularly, to a kit containing:
at least one strip according to the invention, and
a separate container containing the antibiotic with intact β-lactam ring having been used to obtain the antibodies immobilised on the deposition zone 1) of the strip,
optionally, a separate container containing the β-lactamase enzyme and/or a sample not containing β-lactamase enzyme.

### Method of the Invention Using the Strip of the Invention

In a last aspect, the present invention relates to a method for detecting β-lactamases, in a sample capable of containing same. Said sample has been described above.

In a preferred embodiment, said method uses the strip or the kit of the invention containing this strip, as described above.

This method employees the following steps:
a) placing an antibiotic with intact β-lactam ring in contact with the sample to be tested, said antibiotic being specifically recognised by the antibody of the invention, labelled and deposited on the strip, and being immobilised on the TL line of the strip.
b) incubating, for a sufficient time that the enzyme optionally present in the sample can hydrolyse the intact β-lactam ring of the antibiotic,
c) after this incubation, depositing the sample (for example 100 μL) on the deposition zone of the strip, or soaking the strip in the sample and leaving it to react for a sufficient time,
d) reading the result on the test line of the strip.

For the detection with a TL (for example TL: intact antibiotic) (FIG. 5), two cases then arise, depending on what the sample to be tested effectively contains as bacterium or as enzyme (FIG. 6):

Case 1: The sample to be tested contains bacteria which do not produce β-lactamase. After an incubation time, the sample (containing the intact antibiotic and the bacteria) is deposited on the deposition zone of the strip. The antibiotic not having been hydrolysed (because there is no activated β-lactamase in the sample), a complex forms between it and the antibody of the invention. Having migrated to the test line, the antibody complexed in this way cannot bind on the antibiotic which is immobilised there. By contrast, the antibodies will be immobilised at the control line (CL) by the anti-antibody antibody of the invention. Hence only the CL will be visible. The test will be negative and it can be concluded that there are no β-lactamase-producing bacteria in the sample to be tested.

Case 2: The sample to be tested contains bacteria producing a β-lactamase enzyme. After an incubation time, the sample no longer contains intact antibiotic since the β-lactamase enzyme has hydrolysed it. When it is deposited on the deposition zone of the strip, no complex forms between the hydrolysed antibiotic and the antibody of the invention. The antibody of the invention, for which the bond sites are free, migrates towards the test line where it can bind on the intact antibiotic immobilised at the test line. The excess antibody of the invention is itself immobilised at the control line (CL) by the anti-antibody antibody. In this case, the two lines (the test line and the control line) are visible. The test is declared positive, concluding the presence of a bacteria producing β-lactamase (ESBL, or other β-lactamase) in the sample.

For the detection with two TL (for example TL1: intact antibiotic; TL2: anti-CTX-M) (FIG. 10), four cases then occur, depending on what the bacteria present produces as β-lactamase (FIG. 11):

Case 1: The sample to be tested contains bacteria which do not produce β-lactamase. After an incubation time, the sample (containing the intact antibiotic and the bacteria) is deposited on the deposition zone of the strip. The antibiotic not having been hydrolysed (because there is no activated β-lactamase in the sample), a complex forms between it and the antibody of the invention. The labelled anti-CTX-M antibodies do not bind the enzyme CTX-M. Having migrated to test line 1 (TL1), the antibody of the invention thus complexed will not be able to bind itself on the antibiotic which is immobilised there. The labelled anti-CTX-M antibodies not being complexed with the enzyme, they will not be able to be bound by the second anti-CTXM antibody on the test line 2 (TL2). By contrast, the antibodies will be immobilised at the control line (CL) by the anti-antibody antibody. Hence, only the CL will be visible. The test will be negative and it can be concluded that there are no β-lactamase-producing bacteria in the sample to be tested.

Case 2: The sample to be tested contains β-lactamase-producing bacteria but no CTX-M enzyme. After an incubation time, the sample contains no more intact antibiotic since the β-lactamase enzyme has hydrolysed it. When the sample is deposited on the deposition zone of the strip, no complex forms between the hydrolysed antibiotic and the antibody of the invention. The labelled anti-CTX-M antibodies do not bind CTX-M enzyme. The antibody of the invention, for which the bond sites are free, migrates towards the test line where it can bind on the intact antibiotic immobilised at the test line 1 (LT1). The labelled anti-CTX-M antibodies not being complexed with the enzyme, they will not be able to be bound by the second anti-CTXM antibody on the test line 2 (TL2). The anti-CTX-M antibodies and the antibody of the invention in excess are themselves immobilised at the control line (CL) by the anti-antibody antibody. In this case, test line 1 and the control line are visible. The test is declared positive, concluding the presence of a bacteria producing a β-lactamase (ESBL, or other β-lactamase), but which is not a CTX-M enzyme capable of hydrolysing the antibiotic in the sample.

Case 3: The sample to be tested contains bacteria producing a CTX-M type β-lactamase. After an incubation time, the sample contains no more intact antibiotic, since the β-lactamase enzyme has hydrolysed it. When the sample is deposited on the deposition zone of the strip, no complex forms between the hydrolysed antibiotic and the antibody of the invention. The anti-CTX-M antibodies bind the enzyme CTX-M that is present. The antibody of the invention, the bond sites of which are free, migrates towards the test line where it can bind on the intact antibiotic immobilised at the test line 1 (TL1). The anti-CTX-M antibodies present on the test line 2 (TL2) bind the CTX-M enzyme complexed with the labelled anti-CTXM antibodies. The anti-CTX-M antibodies and the antibody of the invention in excess are immobilised at the control line (CL) by the anti-antibody antibody. In this case, the test 1, test 2 and control lines are visible. The test is declared positive, concluding the presence of a bacteria producing a CTX-M type β-lactamase (ESBL or other β-lactamase), capable of hydrolysing the antibiotic in the sample.

In step a) of this method, the antibiotic in intact form (or “substrate”) is added to the sample to be tested. The quantity of intact antibiotic can be adjusted so that the sensitivity of the test is optimal. In order that the test of the invention is functional, it is necessary that the quantity of substrate added to the sample enables the occupation of all the bond sites of the antibodies used, without being in excess. Hence, a signal would appear when the quantity of the substrate is no longer sufficient to occupy all of the bond sites of the antibodies of the invention. An excess of substrate does not allow low concentrations of enzyme to be detected or would require long incubation times that are incompatible with a quick and simple test.

A person skilled in the art, monitoring the indications mentioned in the present invention (and in particular in the examples) will be able to determine the quantity of intact antibiotic to be added to the sample in the initial step of the method, so as to enable the occupation of all the bond sites of the antibodies used, without being in excess.

When an anti-cefotaxime antibody according to the invention is used (cf. example 1 below), it is possible to add, for example, between 10 ng/mL and 50 ng/mL of cefotaxime antibiotic in the initial sample.

More generally, for other antibodies having a strong affinity for the intact antibiotic that they specifically recognise (K_{D} between 1 pM and approximately 10 nM), it will be possible to add between 1 ng/mL and 1 μg/mL, between 10 ng/mL and 1 μg/mL, between 50 ng/mL and 1 μg/mL, between 100 ng/mL and 1 μg/mL or between 100 ng/mL and 0.5 μg/mL of intact antibiotic in the initial sample (containing the bacteria producing or not producing a β-lactamase).

The incubation step b) can be carried out at ambient temperature.

The duration of this step can be adjusted so that the sensitivity of the test is optimal. When cefotaxime is used (cf. example 1 below), this incubation step can last between 10 minutes and one hour. Good results have been obtained with a duration of 30 minutes, under the tested conditions.

Generally, for other antibodies having a strong affinity for the intact antibiotic that they specifically recognise (K_{D} between 1 pM and approximately 10 nM), an incubation period between 10 minutes and one hour is optimal.

Once the sample has been placed in contact with the deposition zone, at the start of step c), the antibodies should be allowed the time to migrate to the test and control lines. This migration step can last between 5 and 30 minutes. When cefotaxime is used (cf. example 1 below), good results have been obtained with a duration between 10 and 20 minutes, under the tested conditions.

Step d) of reading the result can therefore be performed, in the case where cefotaxime is used, approximately 40 minutes after placing the sample and the antibiotic in contact.

In general, for other antibodies having a strong affinity for the intact antibiotic that they specifically recognise (K_{D} between 1 pM and approximately 10 nM), the result will be able to be read approximately 10 minutes to 1 hour (preferably between 20 minutes and 40 minutes) after placing the sample in contact with the strip.

All the steps of this method can be carried out at ambient temperature.

In order to be exploitable, the method of the invention must be able to give reliable results in a minimum time, ideally in less than an hour.

The inventors have been able to demonstrate that, under the conditions used in the examples below, the test of the invention has a specificity of 100% and a sensitivity of 100%, which is excellent.

It can be advantageous to prepare the sample to be tested before using the strip of the invention, in particular if the sample is solid.

If the sample is solid (for example soil), it is possible to dilute it by adding a buffer before proceeding to the step of placing it in contact with the antibiotic. This buffer may contain, for example, NaCl, a molecule known to reduce the non-specific interactions (PVP, PVA, BSA) and the detergent (Tween 20). Its pH is preferably 8. The concentration of NaCl is preferably close to 150 mM.

Preferably, a cellular lysis is performed in order to release the β-lactamase enzyme that is possibly contained in the bacteria of the sample and to make its activity visible more quickly. Conventional lysis buffers can be used (cf. example below).

If the sample is liquid (for example a biological fluid), it is possible to dilute it by adding a buffer before proceeding to the step of placing it in contact with the antibiotic. This buffer may contain, for example, NaCl, a protein known to reduce the non-specific interactions (PVP, PVA, BSA) and the detergent (Tween 20). Its pH is preferably 8. The concentration of NaCl is preferably close to 150 mM.

A person skilled in the art knows how to obtain such exploitable samples. There is no upper limit for the bacteria concentration, unlike for other tests of the prior art.

These preparation steps must not affect the activity of the β-lactamase enzyme possibly present in the sample.

### FIGURES

FIG. 1 describes the principle of immunoenzymatic test 1used in the application to select the antibody of interest that is usable in the system of the invention. This test involves non-hydrolysed cefotaxime-biotin (NH), the antibodies from a hybridoma or immunised mouse plasma with cefotaxime and streptavidin-acetylcholinesterase (G4). The acetylcholinesterase reacts with a chromogen in order to produce a coloured product.

FIG. 2 describes the principle of three other immunoenzymatic tests of interest used to select the antibodies of interest usable in the system of the invention.

Test 2 uses: hydrolysed cefotaxime-biotin (H)+antibody of hybridoma or immunised mouse plasma with cefatoxime+streptavidin-G4

Test 3 uses: non-hydrolysed cefotaxime-biotin (NH)+non-hydrolysed cefotaxime(NH) +antibody of hybridoma or immunised mouse plasma with cefatoxime+streptavidin-G4

Test 4 uses: non hydrolysed cefotaxime-biotin (NH)+hydrolysed cefotaxime (H)+antibody of hybridoma or immunised mouse plasma with cefatoxime+streptavidin-G4

FIGS. 3A and 3B represent the competition curves obtained with hydrolysed or non-hydrolysed cefotaxime, and the various monoclonal antibodies considered (solid line: non-hydrolysed cefotaxime; dotted line: hydrolysed cefotaxime). A: non-identical competition curves obtained for three antibodies with non-hydrolysed cefotaxime (solid line) and the hydrolysed cefotaxime (dotted line). B: similar competition curves obtained for five antibodies with non-hydrolysed cefotaxime (solid line) and hydrolysed cefotaxime (dotted line).

FIG. 4 shows the competition curve obtained with hydrolysed or non-hydrolysed cefotaxime, with one of the unselected monoclonal antibodies.

FIG. 5 shows the various elements constituting the conventional strips used for the purposes of analyte detection.

FIG. 6 describes the two cases expected when the sample contains (positive test) or does not contain (negative test) ESBL bacteria or β-lactamase enzymes.

FIG. 7 describes the plastic cassette which can be used to protect the strip of the invention.

FIG. 8 describes the structures of the carbapenem compounds used in example 2 (B).

FIG. 9 shows the principle of tests 1 to 4 which are described in example 2 (C).

FIG. 10 shows a strip according to the invention, containing two test lines and a control line. The first test line corresponds to a zone where cefotaxime-BSA has been immobilised, and the second test line corresponds to a zone where anti-CTX-Ms antibodies have been immobilised. The control line corresponds to a zone where secondary antibodies, recognising the other labelled antibodies used in the invention, have been immobilised. In the deposition zone, anti-cefotaxime antibodies of the invention and anti-CTX-Ms antibodies have been deposited, all labelled with colloidal gold.

FIG. 11 describes the three cases expected when the sample does not contain bacteria having an ESBL or β-lactamase enzymess (negative test), or contains ESBL bacteria or β-lactamase enzymess other than CTX-M (positive test on one line), or contains ESBL bacteria or CTX-M type β-lactamase enzymes(positive test on the two lines).

### EXAMPLE 1

### Detection of Bacteria Resistant to Cefotaxime

A. Design and Production of the Immunogen

Cefotaxime is a small molecule incapable of inducing an immune response, essential for obtaining antibodies. It was therefore necessary to couple this antibiotic to a larger immunogenic molecule, bovine serum albumin (BSA). Since the difference in recognition of the antibodies of the invention should occur at the β-lactam core, a particular immunogen was designed, which enabled optimum exposure of the β-lactam core to the immune system. The coupling with BSA was therefore carried out at the NH₂ function, which is the function furthest from the β-lactam core. The cefotaxime was activated with chloroacetyl chloride (Rodriguez, “An improved Method for preparation of cefpodoxime proxetil”, 2003). To do this, a suspension of cefotaxime (500 mg, 1.09 mmol, 1 eq.) dissolved in 2 ml of DMA, was added to the chloroacetyl chloride (128 μl, 1.65 mmol, 1.5 eq.) at 5° C.-10° C. The mixture was then stirred for 1 hour 30 minutes at ambient temperature. Once the operation was completed, the solution was poured into ice. The precipitate was collected by filtration and washed successively with H₂O, ethanol and diethyl ether, and then dried in order to obtain the desired product in the form of a white-grey powder (349 mg, 0.66 mmol, 60%). This reaction led to the formation of a chloroacetamido function which can react, in particular, with thiol functions.

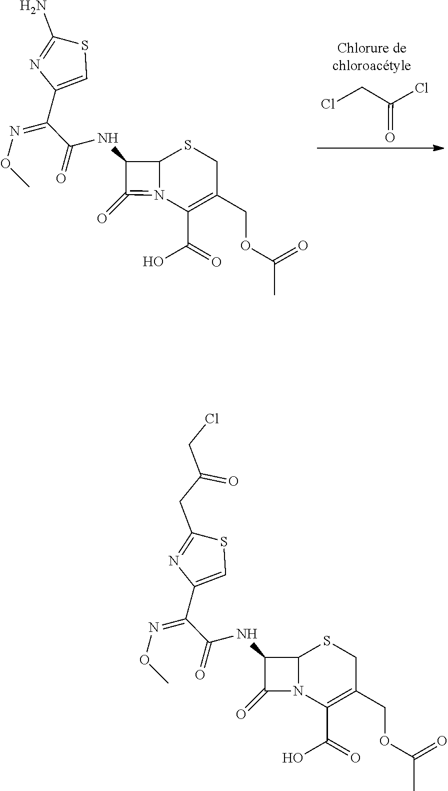

| Céfotaxime | Céfotaxime chloroacétamide |
|---|---|
| French | English |
| Chlorure de chloroacétyle | Chloroacetyl chloride |
| Céfotaxime | Cefotaxime |
| Céfotaxime chloroacétamide | Cefotaxime chloroacetamide |

In parallel, 35 mg of bovine serum albumin (BSA) was dissolved in 1 ml of 0.1 M, pH 7.4 sodium phosphate buffer. 50 μl of a solution of 122 mg/ml of N-succinimidyl-s-acetylthioacetate (SATA) in DMF were added (molar ratio SATA/BSA=50). After a reaction of 16 hours at 4° C., the product was purified by molecular sieve chromatography using a Sephadex G25 medium column. Then, the protection of the thiol function was removed by adding 100 μl of 1 M, pH 7 hydroxylamine for 30 minutes at 20° C. The concentration of thiol was measured (SH/BSA=20.7) by reaction with DTNB. This product can then react with the chloroacetamido function of the modified cefotaxime.

For this reason, 2.34 mg of chloroacetamido-cefotaxime at 6 mg/ml in DMSO was added to 2.76 mg of BSA-SH (molar ratio of chloroacetamido-cefotaxime/SH=5). With a reaction for 1 hour 30 minutes at 20° C., 50 μl of 1 M, pH 9.0 borate buffer was added and incubated for 1 hour 30 minutes. A dialysis was carried out with a dialysis cassette of 3500 MWCO. The concentration of BSA-cefotaxime was then determined by BCA reaction.

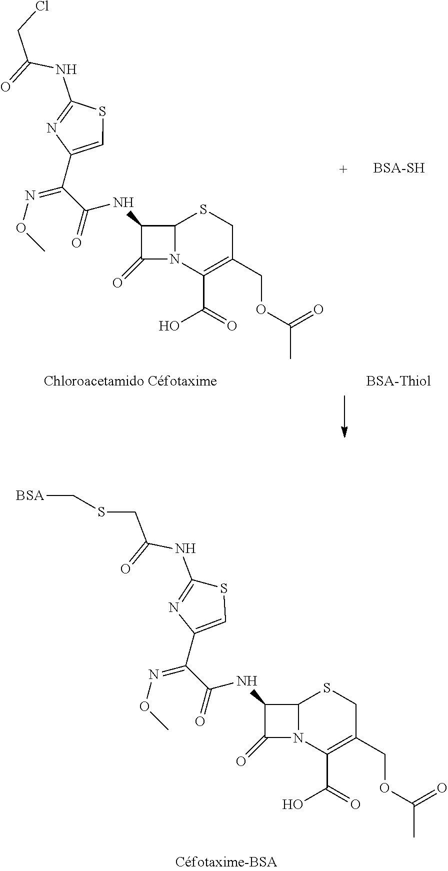

| French | English |
|---|---|
| Chloroactamido Céfotaxime | Chloroactamido-cefotaxime |
| Céfotaxime-BSA | Cefotaxime-BSA |
| BSA-SH | BSA-SH |
| BSA-Thiol | BSA-Thiol |
| Céfotaxime-BSA | Cefotaxime-BSA |

The cefotaxime-BSA was used to immunise mice. In order to carry out the immunisations, subcutaneous injections of 50 μg of cefotaxime-BSA/mouse were carried out every three weeks for three months (4 immunisations in total). After 2 months rest for the mice, new injections of cefotaxime-BSA were carried out intravenously for said mice: 50 μg of product/mouse, once per day for three days. After two days rest, spleen cells of the mouse were fused with NS1 mouse myeloma cells, and anti-cefotaxime specific antibodies in myeloma culture supernatants were detected using an immunoenzymatic test.

B. Production and Purification of the Various Forms of Cefotaxime For the proper implementation of the invention, it is essential that the difference in affinity of the antibodies of the invention for the intact and hydrolysed form of the antibiotic is maximal. To do this, it was necessary to have non-hydrolysed cefotaxime and hydrolysed cefotaxime. On the other hand, it was also necessary to have available ‘tracer’ molecules enabling the specific antibodies to be detected, which are non-hydrolysed cefotaxime-biotin and hydrolysed cefotaxime-biotin. These molecules can be detected by reaction with acetylcholinesterase-streptavidin (G4). Acetylcholinesterase reacts with the chromogen to produce a coloured substrate.

Production of Non-Hydrolysed and Hydrolysed Cefotaxime-Biotin

Non-hydrolysed cefotaxime-biotin was obtained by coupling chloroacetamido-cefotaxime and biotin coupled to a polyethylene glycol (PEG) arm and a thiol function (Biotin-PEGx-Thiol), by using the procedure described above for the immunogen. Chloroacetamido-cefotaxime (31.6 mg, 0.06 mmol, 1 eq.) and Biotin-PEGx-Thiol (94 mg, 0.119 mmol, 2 eq.) were dissolved in 0.5 ml of DMF and 2 μl of triethylamine, and with then added to the mixture under argon. The reaction was stirred for 3 days. Once the reaction was finished, the mixture was evaporated under reduced vacuum. Then, the product was purified by reversed-phase chromatography on a water/acetonitrile gradient of 0 to 40% (peak isolated at 26% acetonitrile). The molecular weight of this tracer was checked by mass spectrometry, where a purification cycle of 15 minutes on a C18 column is carried out, then the sample is ionised on a quadrupole.

The hydrolysed cefotaxime-biotin was obtained by enzymatic reaction with beads coupled to KPC-2 (Klebsiella pneumoniae carbapenemase), which is a recombinant β-lactamase. To do this, 5 mg of beads (Dynabeads M-280 Tosylactivated) were washed with the 0.1 M borate buffer, pH 9.5. 100 μg of the recombinant protein KPC-2 were added to the beads in a volume of 150 μl. Then, 100 μl of 0.1 M pH 9.5 borate buffer 0+3 M ammonium sulfate were added. After 16 hours of reaction at 37° C., 1 ml of 0.1 M pH 7.4 sodium phosphate buffer+0.15 M sodium chloride+0.5% BSA were added. After 1 hour of reaction at 37° C., the coupling product was washed with the 0.1 M pH 7.4 sodium phosphate buffer+0.15 M sodium chloride+0.1% BSA and concentrated to 20 mg/ml of beads. The enzymatic activity of this product was tested with nitrocefin. For this, 20 μl of 0.5 mM nitrocefin were added to a solution of 10 μg/ml of Beads-KPC-2, diluted in a 50 mM pH 7.4 sodium phosphate buffer, in a total volume of 200 μl. After 30 minutes reaction at 20° C., the absorbance is measured at 492 nm. Subsequently, starting from the non-hydrolysed cefotaxime biotin, 50 μl of the Billes-KPC-2 solution at 20 mg/ml was added to 1 ml of a solution of non-hydrolysed cefotaxime-biotin at 2 mg/ml. After reaction for 16 hours at 25° C., the Beads-KPC-2 were removed using a magnet. The supernatant was recovered and purified by reversed-phase chromatography on a water/acetonitrile gradient of 0 to 40% (peak isolated at 23% acetonitrile). The molecular weight of this tracer was checked by mass spectrometry, where a purification cycle of 15 minutes on a C18 column is carried out, then the sample is ionised on a quadrupole.

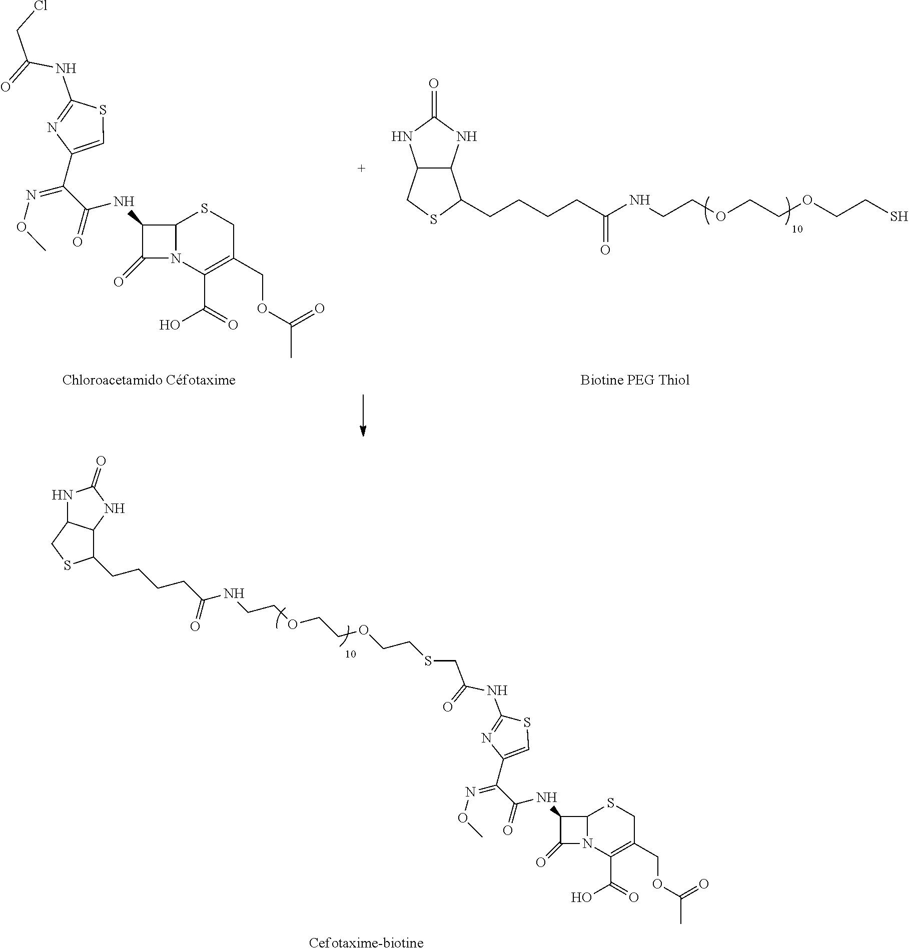

| French | English |
|---|---|
| Chloroactamido Céfotaxime | Chloroactamido-cefotaxime |
| Biotine PEG Thiol | Biotin PEG Thiol |
| Céfotaxime-biotine | Cefotaxime-biotin |

Production of Non-Hydrolysed and Hydrolysed Cefotaxime

Non-hydrolysed cefotaxime (Sigma-Aldrich) was then purified by reversed-phase chromatography on a water/acetonitrile gradient of 0 to 20% (peak isolated at 8.5% acetonitrile). The molecular weight of this product was checked by mass spectrometry, where a purification cycle of 15 minutes on a C18 column is carried out, then the sample is ionised on a quadrupole.

The hydrolysed cefotaxime was also obtained by enzymatic reaction with beads coupled to KPC-2. The same beads-KPC-2 coupling protocol, cited above, was carried out. Then, starting from the non-hydrolysed cefotaxime, 50 μl of the Beads-KPC-2 solution at 20 mg/ml were added to 1 ml of a solution of non-hydrolysed cefotaxime at 2 mg/ml. After reaction for 16 hours at 25° C., the Beads-KPC-2 were removed using a magnet and the solution was purified by reversed-phase chromatography on a water/acetonitrile gradient of 0 to 20% (peak isolated at 2.5% acetonitrile). The molecular weight of this tracer was checked by mass spectrometry, where a purification cycle of 15 minutes on a C18 column is carried out, then the sample is ionised on a quadrupole.

Purification of the Various Forms of Cefotaxime

All of the solutions were purified by reversed-phase chromatography on a water/acetonitrile gradient of 1 ml/ml. For the non-hydrolysed cefotaxime, the isolated peak is at 8.5% acetonitrile. For the hydrolysed cefotaxime, the peak is at 2.5%. For the non-hydrolysed cefotaxime-biotin, it is at 26%, whereas for the hydrolysed cefotaxime-biotin, the isolated peak is at 23.5%. All of these 4 compounds were characterised by mass spectrometry. For this, they are purified in a 15-minute cycle on a C18 column (water/acetonitrile gradient), then ionised in a quadruple. The molecular weight of each compound was identified: m/z of non-hydrolysed cefotaxime=456; m/z of hydrolysed cefotaxime=414; m/z of non-hydrolysed cefotaxime-biotin=1241.5; m/z of hydrolysed cefotaxime-biotin=1283.5. The following compounds were obtained:

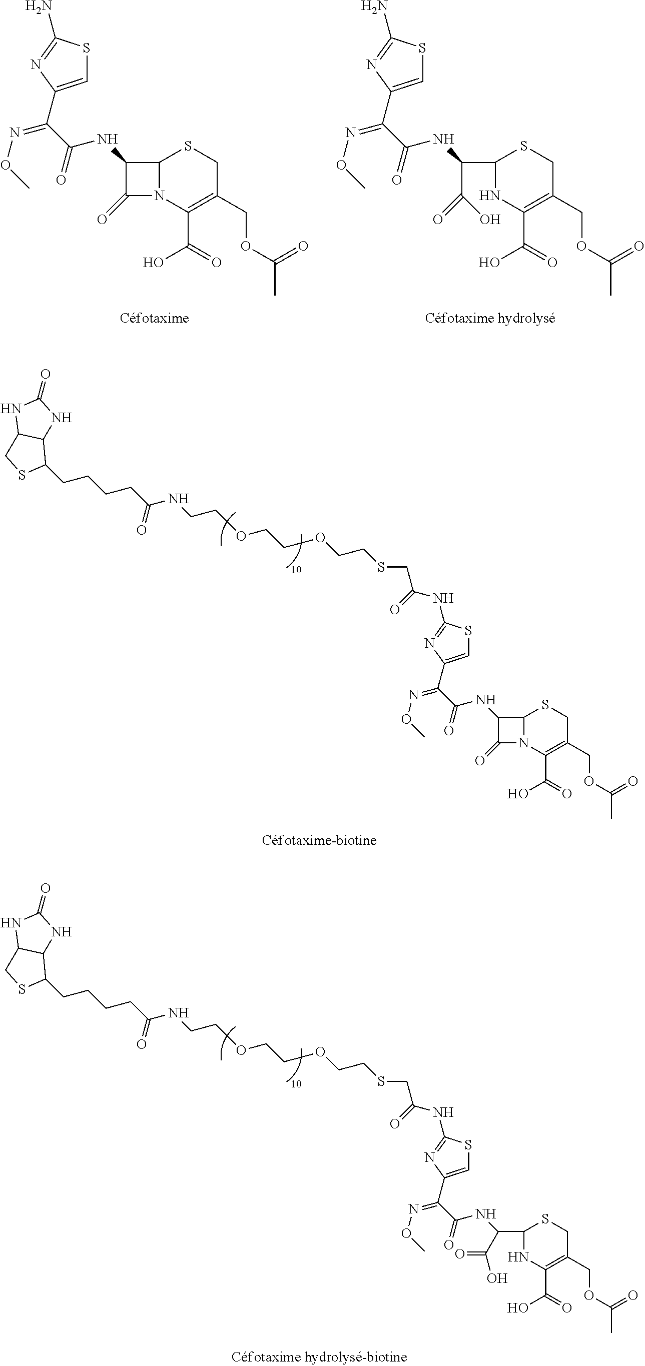

| French | English |
|---|---|
| Céfotaxime | Cefotaxime |
| Céfotaxime hydrolysé | Hydrolysed cefotaxime |
| Céfotaxime-biotine | Cefotaxime-biotin |
| Céfotaxime hydrolysé-biotine | Hydrolysed cefotaxime-biotin |

C. Production and Selection of the Antibodies of Interest

Four mice were immunised with cefotaxime-BSA. In order to do this, subcutaneous injections of 50 μg of cefotaxime-BSA/mouse were carried out every three weeks for three months (4 immunisations in total). After 3 months rest for the mice and in order to select the mice having the best immune response, their antibodies were analysed with a first test. In this test, the murine antibodies taken during the immunisation protocol were captured by a first murine anti-antibody antibody (AffiniPure Goat Anti-Mouse IgG+IgM (H+L); Jackson Immunoresearch LABORATORIES) immobilised on the wall of wells of a microtitration plate. 100 μl at 100 ng/ml of non-hydrolysed cefotaxime-biotin was added in each well. After incubation at 4° C. overnight and after washing, 100 μl of streptavidin-G4 at 1 EU/ml was added in order to reveal the presence of intact cefotaxime coupled with biotin and therefore the presence of non-hydrolysed anti-cefotaxime antibodies. Acetylcholinesterase (G4) activity was measured by the Ellman method (Ellman et al., 1961). The Ellman medium comprises a mixture of 7.5 10⁻⁴ M acetylthiocholine iodide (enzymatic substrate) and 2.5 10⁻⁴ M 5,5′-dithiobis(2-nitrobenzoic acid) (DTNB) (reagent for the calorimetry measurement of thiol) in a 0.1 M pH 7.4 phosphate buffer. The enzymatic activity is expressed in Ellman units (EU). One EU is defined as the quantity of enzyme producing an increase in absorbance of one unit during 1 minute in 1 ml of medium, for an optical path length of 1 cm: it corresponds to approximately 8 ng of enzyme.

After one hour incubation at ambient temperature and after washing, 200 μl of Ellman medium reagent are added to the wells. After 30 minutes and/or one hour, the signal intensities are measured. The intensity of the signal is obtained during this test is then proportional to the quantity of non-hydrolysed cefotaxime specific antibodies. The mice having the best immune response (larger concentration of specific antibodies) then received new injections of cefotaxime-BSA. For this, the product was administered intravenously to the mice with the best responses: 50 μg of product/mouse, once per day for three days. After two days of rest, they were sacrificed and their splenocytes (spleen cells) were hybridised with NS1 mouse myeloma cells in order to obtain hybridomas (producers of antibodies and immortal cells) (Grassi, J., Frobert, Y., Lamourette, P. and Lagoutte, B., 1988. “Screening of monoclonal antibodies using antigens labeled with acetylcholinesterase: application to the peripheral proteins of photosystem 1”. Anal. Biochem. 168, 436).

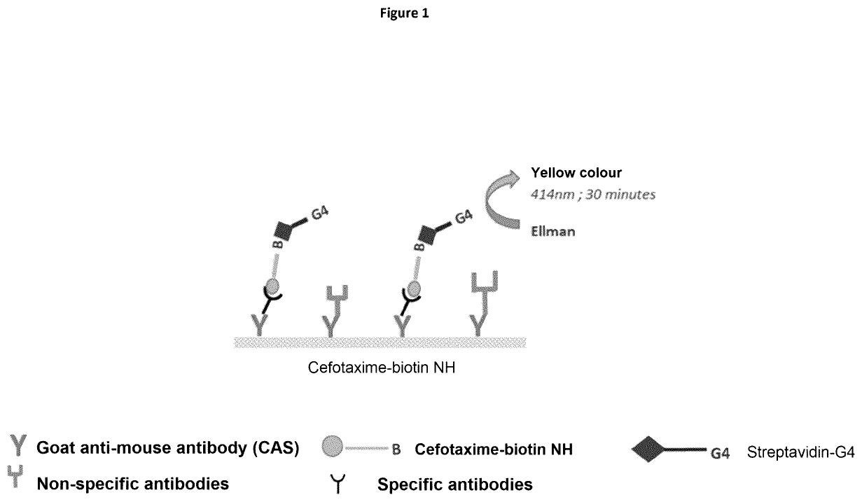

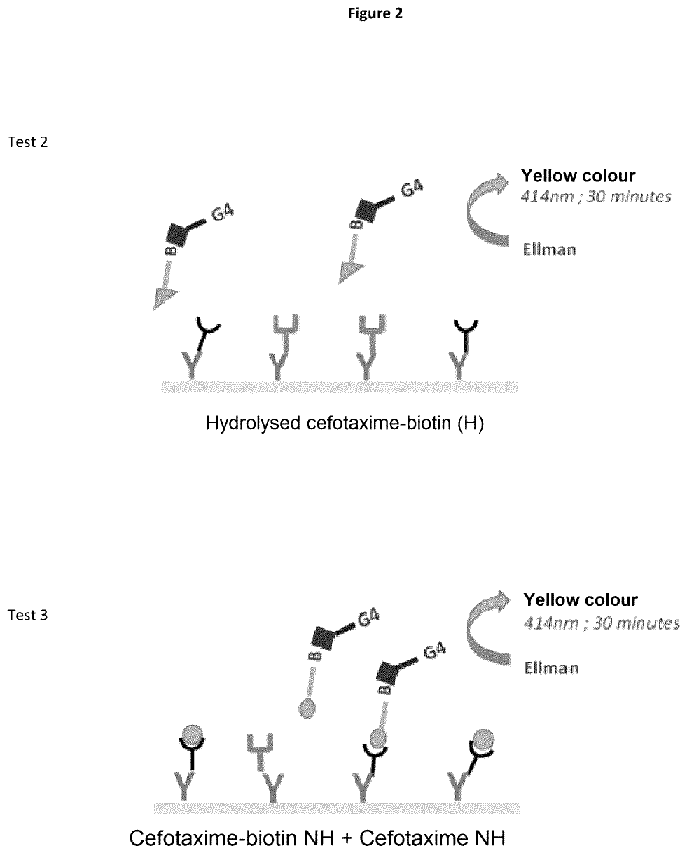

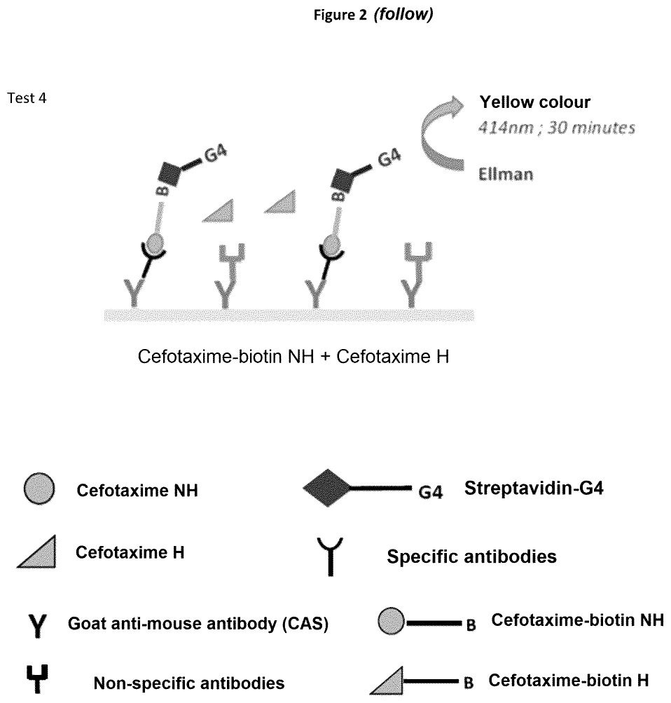

At the end of the fusion, all of the cells were distributed into the wells of 10 microtitration plates. After one week, the presence of antibodies recognising cefatoxime in each well was analysed using test 1 (FIG. 1). This test could select and preserve the cells of 107 wells. In order to refine this selection and keep only the hybridomas producing antibodies that recognise only non-hydrolysed cefotaxime, the culture supernatants of the selected wells were analysed using 4 different tests (FIGS. 1 and 2: tests 1 to 4):

Test 1: In this test, the antibodies present in the culture supernatants are captured by a first murine anti-antibody antibody immobilised on the wall of wells of a microtitration plate. The non-hydrolysed cefotaxime-biotin is added to each well. After incubation at 4° C. overnight and after washing, streptavidin-G4 is added in order to reveal the presence of non-hydrolysed cefotaxime coupled with biotin and therefore non-hydrolysed anti-cefotaxime antibodies.

Test 2: In this test, the antibodies present in the culture supernatants are captured by a first murine anti-antibody antibody immobilised on the wall of wells of a microtitration plate. Hydrolysed cefotaxime-biotin is added to each well. After incubation at 4° C. overnight and after washing, streptavidin-G4 is added in order to reveal the presence of hydrolysed cefotaxime coupled with biotin and therefore the presence of hydrolysed anti-cefotaxime antibodies.

Test 3: In this test, non-hydrolysed cefotaxime-biotin is placed in competition with non-hydrolysed cefotaxime with respect to recognition by the specific antibodies present in the culture supernatants. After incubation at 4° C. overnight and after washing, streptavidin-G4 is added in order to reveal the presence of intact cefotaxime coupled with biotin.

Test 4: In this test, non-hydrolysed cefotaxime-biotin is placed in competition with hydrolysed cefotaxime at the same concentration as the non-hydrolysed cefotaxime used in test 3, with respect to recognition by the specific antibodies present in the culture supernatants. After incubation at 4° C. overnight and after washing, streptavidin-G4 is added in order to reveal the presence of intact cefotaxime coupled with biotin.

For tests 1 and 2, the appearance of the signal in the wells indicates the presence of non-hydrolysed anti-cefotaxime antibodies and hydrolysed anti-cefotaxime antibodies respectively.

For tests 3 and 4, a reduction in the signals proportional to the concentration of inhibitor reveals the presence of antibodies recognising the inhibitor: non-hydrolysed cefotaxime (test 3) or hydrolysed cefotaxime (test 4). On the other hand, these tests make it possible to evaluate the relative specificity of the antibodies for non-hydrolysed cefotaxime and hydrolysed cefotaxime. Hence, if the reduction in the signal is similar for the two forms of cefotaxime, then the antibodies have the same affinity for these two molecules. If the reduction in the signal is weaker for one of the two forms of cefotaxime, then the antibodies have a weaker affinity for this form.

The wells were selected for which a signal is obtained for test 1 and no signal for test 2, a largest signal reduction of the signal for test 3 and no reduction of the signal for test 4. At the end of the selection process, 18 hybridomas were preserved in order to produce monoclonal antibodies.

D. Characterisation of the Monoclonal Antibodies

In order to evaluate the specificity of each monoclonal antibody, tests 3 and 4 were carried out with various concentrations of non-hydrolysed cefotaxime and hydrolysed as a competitor. The specificity was determined by calculating the percentage of cross-reaction between the two forms of cefotaxime. In order to carry out this calculation, the concentration of non-hydrolysed cefotaxime was divided by the concentration of hydrolysed cefotaxime which causes the same reduction in signal. For example, if a reduction in the signal is induced by 1 nmol/ml of non-hydrolysed cefotaxime and by 100 nmol/ml of hydrolysed cefotaxime, then the percentage of cross-reaction is: 1/100=0.01 therefore 1%.

In order to carry out these tests, all of the solutions below have been prepared in a buffer having, for composition: 0.1 M pH 7.4 potassium phosphate buffer+0.1% PVP+0.15 M NaCl+0.01% sodium azide. Intact cefotaxime-biotin was used at 0.3 pmol/ml. For non-hydrolysed cefotaxime and hydrolysed cefotaxime, a range of concentrations was produced: 210 pmol/ml; 21 pmol/ml; 2.1 pmol/ml; 0.21 pmol/ml and 0 pmol/ml

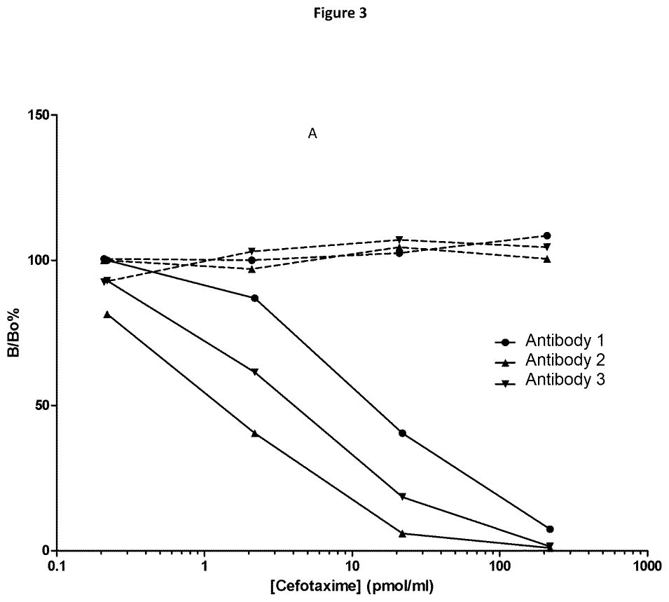

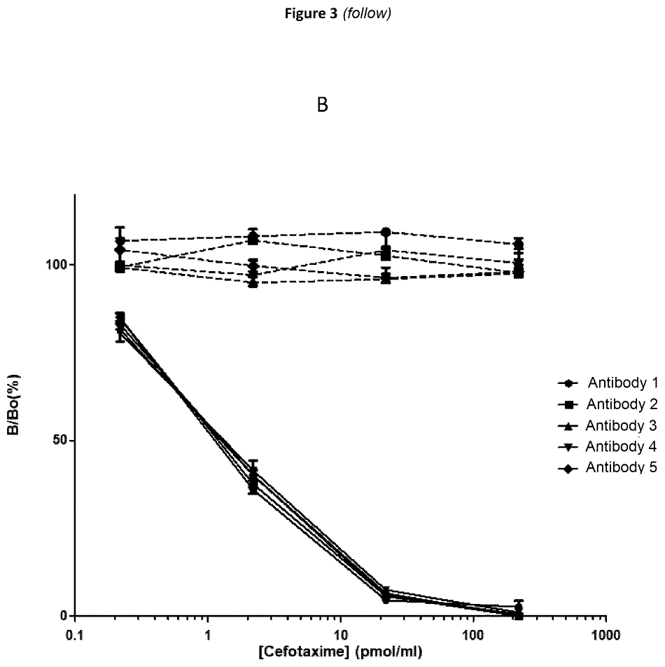

The solutions were deposited on a 96-well microplate, on the wall of which a mouse anti-antibody antibody (identical to that used in the experiments for selecting antibodies) was immobilised beforehand, at a level of 25 μl of marker (intact cefotaxime-biotin) and 25 μl of competitor (non-hydrolysed or hydrolysed cefotaxime). Then 50 μl of the antibody solution was added. The microplates were incubated overnight at 4° C., then, after washing, 100 μl of streptavidin-G4 was added for 1 hour at ambient temperature and under stirring. After washing the wells, 200 μl of chromogen (Ellman medium) was deposited. The reading of the absorbance was carried out after 1 hour of incubation under stirring, at 414 nm by the spectrophotometer. The graphs obtained are f([Cefotaxime])=% B/Bo. The signal Bo corresponds to the absorbance obtaining in absence of competitor (maximum absorbance). The signal B is the absorbance in the medium where competitor and marker interact with the antibodies. In order that the figure is readable, only the results obtained with several antibodies is shown (FIGS. 3A and 3B).

For 16 antibodies, no reduction in the signal was observed with the strongest concentration of hydrolysed cefotaxime (210 pmol/ml). The cross-reactions are therefore less than 0.1% (minimum concentration of non-hydrolysed cefotaxime inducing a signal reduction/210 pmol/ml, multiplied by 100). For the two other antibodies, a small reduction in the signal was observed and the cross-reactions obtained are only 0.008% and 0.045%.

It can be seen in FIG. 3A that the competition curves obtained with non-hydrolysed cefotaxime with the various antibodies are not identical. Whereas, for other antibodies, the competition curves obtained with non-hydrolysed cefotaxime are similar (FIG. 3B).

It can therefore be seen that the competition curves obtained with non-hydrolysed cefotaxime with the various antibodies are not identical. This means that the the antibodies involved are different. Hence, the specificity of the antibodies of the invention is not linked to a particular protein sequence of the bonding site of these antibodies. It is the design of the immunogen and the selection strategy chosen which makes it possible to select such antibodies.

Among these 18 antibodies, that which has the largest affinity for non-hydrolysed cefotaxime (antibody 3) was selected for the development of the activated cephalosporinase detection test.

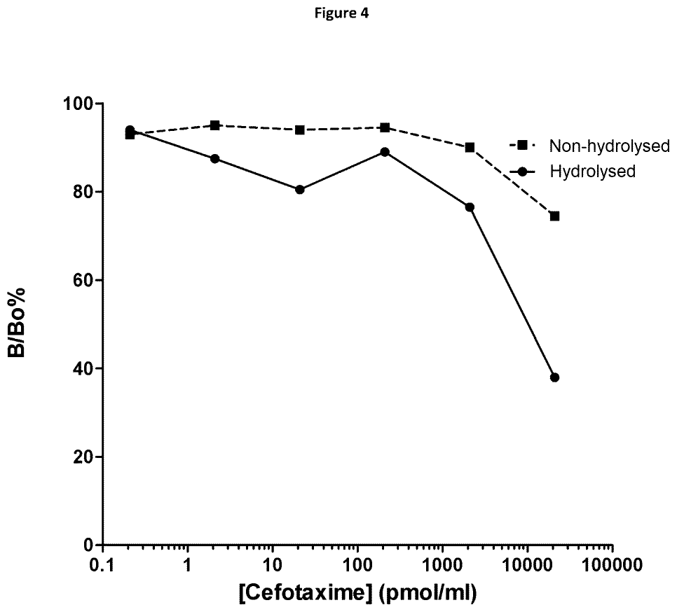

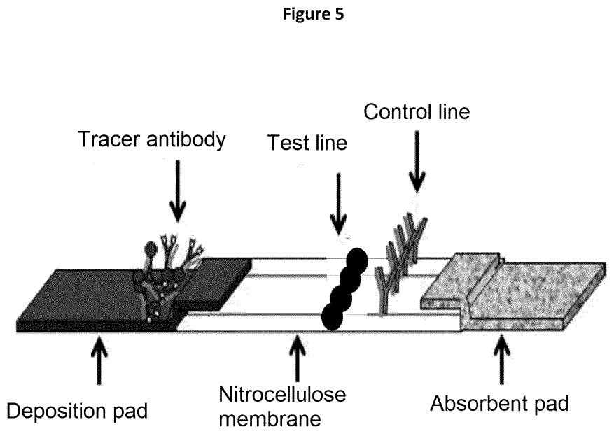

The specificity of an antibody that was not retained at the end of the selection has also been analysed. FIG. 4 shows, for this unretained antibody, a larger reduction in the signal for hydrolysed cefotaxime than for non-hydrolysed cefotaxime, which means that the latter form is less well recognised by the unretained antibody.

E. β-Lactamase Activity Detection Test According to the Invention

The selected antibodies (Antibody 1; 2; 3; 4 and 5) were then used on tests strips by competition.

As described above, the strips consist of four distinct parts:
1) The sample paper (SP) for depositing the sample.
2) The conjugated paper (CP) whether labelled antibody (also called tracer) is dried. This paper can be an accessory in the case where the tracer antibody is used in liquid format. In this case, 10 μl of tracer antibody is added to 100 μl of sample before the deposition on the strip.
3) The nitrocellulose membrane (MbNC) with two lines (Test Line (TL): BSA-intact cefotaxime; Control Line (CL): anti-antibody antibody tracer).
4) The absorbent paper (AP) for enabling the migration of the sample through the strip.

Parameters of the Test Strip

All of the solutions below have been prepared in the buffer strip, having the composition: 0.1 M pH 8 Tris buffer/HCL+0.15 M NaCl+0.5% Tween 20+1% chaps+0.01% sodium azide. It enables lysis of bacteria and the release of its content (enzymes, for example).

The following three parameters have been optimised:
The quantity of tracer: The chosen antibody is coupled to colloidal gold (coloured marker), its absorbance could be measured at 530 nm. For this reason, in the results, the term absorbance (DO) relative to colloidal gold is employed. Several DO were therefore tested. The objective was to determine the smallest quantity of tracer enabling a significant signal to be observed 10 minutes after depositing of the sample on the strip.
The quantity of BSA-intact cefotaxime on the TL: once the DO in antibodies is fixed, several concentrations of BSA-intact cefotaxime on the TL were tested (1 μL/cm). The objective was to fix the non-limiting lowest concentration for the observed signal at the TL.

The optimisation of these two parameters was carried out for the five antibodies selected. However, to simplify the figures, only the results obtained with antibody 3 have been reported.
The quantity of non-hydrolysed cefotaxime to be added to the samples: once the two preceding parameters are determined, the objective was to fix the minimum concentration of intact cefotaxime from which no signal is observed on the TL. In parallel, hydrolysed cefotaxime was added in order to check that the antibody is indeed specific to the non-hydrolysed form. For this, a range of non-hydrolysed cefotaxime and a range of hydrolysed cefotaxime were prepared with the buffer strip: 1000 ng/ml; 100 ng/ml; 10 ng/ml; 1 ng/ml.

For all these three parameters, a colour intensity scale was used to evaluate the results obtained on the test strips. This scale was defined from 1 to 10, where each value is characteristic of an increasing signal intensity.

96-well microplates were used for all these tests. In order to start a test, 10 μl of tracer in liquid form was added to 100 μl of buffer containing or not containing cefotaxime. Then, a strip composed of a sample paper, a nitrocellulose membrane and an absorbent paper, were deposited in the wells. An incubation of 10 minutes was performed, then the signal intensity was evaluated with the colour intensity scale.

### Results

The DO of the tracer was first optimised. A default concentration of 1 mg/ml of BSA-intact cefotaxime was deposited on the TL. The results of the tests are given in Table 1.

TABLE 1
Optimisation of the DO of the tracer.

| | Signal |
|---|---|
| DO used | intensity |
| DO:8 | 10 |
| DO:4 | 10 |
| DO:2 | 9 |
| DO:1 | 8.5 |
| DO:0.5 | 7.5 |

In order to obtain a sufficient signal intensity, DO:1 was selected because it is located in the intensity scale 8.5/9.

Various concentrations of BSA-non-hydrolysed cefotaxime were then deposited on the TL (1 μL/cm). The concentration DO: 1 previously selected for the tracer has been used.

TABLE 2
Optimisation of the concentration of BSA-cefotaxime on the TL.

| Concentration of BSA- | | Signal intensity |
|---|---|---|
| intact cefotaxime | | DO:1 |
| 1 | mg/ml | 9.5 |
| 0.6 | mg/ml | 10 |
| 0.3 | mg/ml | 10 |
| 0.1 | mg/ml | 9 |
| 0.03 | mg/ml | 8 |

The intensity of the signal only increases very weakly beyond a concentration of 0.1 mg/ml in BSA-cefotaxime. This is why this concentration was selected. In order to more precisely adjust the parameters, various quantities of tracer were tested with this quantity of BSA-cefotaxime.

TABLE 1
Second optimisation of the DO of tracer.

| Concentration of | |
|---|---|
| BSA-intact | Signal |
| cefotaxime | intensity |
| DO:1 | 9.5 |
| DO:0.5 | 8.5 |
| DO:0.25 | 7.5 |
| DO:0.125 | 5.5 |

In view of these results, the concentration of 0.1 mg/ml of BSA-cefotaxime on the TL and the tracer DO 0.5 were kept, because the signal obtained was around 8.5.

The concentration of non-hydrolysed cefotaxime to be used in the samples was then determined. A control was produced (condition at 0 ng/ml), in order to see the maximum signal that could be obtained on the TL (Table 4).

TABLE 4
Optimisation of the concentration of intact cefotaxime to be
added to the samples. Ø: no signal was observed on the TL.

| | | Signal intensity | |
|---|---|---|---|
| | | Non- | |
| Cefotaxime | | hydrolysed | Hydrolysed |
| concentration | | cefotaxime | cefotaxime |
| 1000 | ng/ml | Ø | 8 |
| 100 | ng/ml | Ø | 9 |
| 10 | ng/ml | Ø | 9 |
| 1 | ng/ml | 4 | 9 |
| 0 | ng/ml | 9 | 9 |

For the non-hydrolysed cefotaxime, the concentration from which the signal is visible on the TL is 1 ng/ml. The lowest concentration enabling a total disappearance of the signal (all the recognition sites of the tracer antibodies occupied) is therefore 10 ng/ml.

For the hydrolysed cefotaxime, the signal is equivalent to the control (the tracers bind themselves on the TL). As expected, the hydrolysed cefotaxime is not recognised by the tracer antibody for which the bond sites are free to integrate with the cefotaxime of the TL.

The concentration 10 ng/ml of BSA-cefotaxime was then used to study the kinetics of cefotaxime hydrolysis. For the same concentration of hydrolysed cefotaxime, no reduction in the signal was observed. The hydrolysis of cefotaxime in the sample well therefore leads to an increase in the signal at the TL.

By way of example, other antibodies have been tested. The conditions of use of the antibodies are as follows: Antibody 1, DO0.5, 0.3 mg/ml of BSA-cefotaxime; Antibody 2, DO1, 0.3 mg/ml of BSA-cefotaxime; Antibody 3, DO0.5, 0.1 mg/ml of BSA-cefotaxime; Antibody 4, DO0.5, 0.3 mg/ml of BSA-cefotaxime; Antibody 5, DO0.5, 0.3 mg/ml of BSA-cefotaxime (Table 5).

TABLE 5
Optimisation of the concentration of intact cefotaxime to be added to the
samples for different antibodies. Ø: no signal was observed on the TL.

| | | Antibody 1 | | Antibody 2 | | Antibody 3 |
|---|---|---|---|---|---|---|
| | | NH | H | NH | H | NH |
| Inhibition | | cefotaxime | cefotaxime | cefotaxime | cefotaxime | cefotaxime |
| 1000 | ng/ml | Ø | 7.5 | Ø | 8.5 | Ø |
| 100 | ng/ml | Ø | 9 | Ø | 8.5 | Ø |
| 10 | ng/ml | Ø | 9 | 4 | 8.5 | Ø |
| 1 | ng/ml | 5 | 9 | 8 | 8.5 | 4 |
| 0 | ng/ml | 9 | | 8.5 | | 9 |

| | | |
|---|---|---|
| Antibody 3 | Antibody 4 | Antibody 5 |

| | H | NH | H | NH | H |
|---|---|---|---|---|---|
| Inhibition | cefotaxime | cefotaxime | cefotaxime | cefotaxime | cefotaxime |

| | | | | | | |
|---|---|---|---|---|---|---|
| 1000 | ng/ml | 8 | Ø | 7 | Ø | 7.5 |
| 100 | ng/ml | 9 | Ø | 9 | Ø | 9 |
| 10 | ng/ml | 9 | Ø | 9 | Ø | 9 |
| 1 | ng/ml | 9 | 4.5 | 9 | 5 | 9 |
| 0 | ng/ml | | 9 | | 9 | |

In view of these results, the Antibody 3 showed the best performance under the strip format because it made it possible to detect an enzymatic activity for the weakest concentration of enzyme. However, this result shows that other antibodies would have been able to be used in the context of this method. For the rest of the developments and optimisations, only Antibody 3 was used.

Hydrolysis kinetics with a recombinant enzyme.

Once the parameters of the test strip were defined, the hydrolysis kinetics were realised. For this study, the enzyme CTXM-2, an ESBL from Escherichia coli was used. The objective of these kinetics is to succeed in obtaining a positive signal “+” (signal visible on the TL) as soon as possible, and at the lowest possible enzyme concentration.

In order to carry out these tests, all of the solutions below have been prepared in the buffer strip having, for composition: 0.1 M pH 8 Tris buffer/HCL+0.15 M NaCl+0.5% Tween 20+1% chaps+0.01% sodium azide. It enables lysis of bacteria and the release of its content.

The concentrations used are: 10 ng/ml; 3 ng/ml; 1 ng/ml; 0.3 ng/ml; 0.1 ng/ml enzymes, and are incubated with intact cefotaxime at ambient temperature. Two controls are also produced containing either only intact cefotaxime (no signal should be observed on the TL because all of the tracer binding sites are occupied), or only the buffer strip (maximum signal able to be obtained on the TL because all of the tracer binding sites are free).

In this study, in a first step the tracer is added in liquid form (10 μl at DO:0.5 are added to 100 μl of the enzymatic solution) then, in a second step, in dried format on a conjugated paper (10 μl at DO:0.9). The conjugated paper was then inserted on the strip between the sample paper and the nitrocellulose membrane. For this reason, two types of strips were used, with or without conjugated paper (CP). For each experiment, it was indicated which type of strip had been used. The TL on the nitrocellulose membrane is composed of 0.1 mg/ml of BSA-non-hydrolysed cefotaxime. The various conditions were prepared then incubated at ambient temperature. Once the incubation time had expired, 100 μl of solution was sampled and deposited, either in a microplate well or in a deposition well of a plastic cassette.

The samples were tested after 0 minutes; 15 minutes; 30 minutes; 45 minutes; 60 minutes incubation. The reading on the test strip was made after 10 minutes migration. A signal on the TL is considered as “P”. An absence of signal on the TL is defined as “N”. A first hydrolysis kinetics was produced at ambient temperature. The tests were carried out on 96-well microplates, with 100 μl of sample+10 μl of tracer in liquid format. The strips without CP were deposited in the wells. The concentration of intact cefotaxime is 10 ng/ml, with a tracer DO of 0.5 (Table 6).

TABLE 6
Hydrolysis kinetics at ambient temperature of cefotaxime by a CTXM-2 enzyme, liquid tracer
(N: negative test no signal at the TL; P: positive test, signal visible at the TL).

| | 0 | 15 | 30 | 45 | 60 |
|---|---|---|---|---|---|
| Concentration of CTXM-2 | minutes | minutes | minutes | minutes | minutes |
| 10 ng/ml | N | P | P | P | P |
| 3 ng/ml | N | N | N | P | P |
| 1 ng/ml | N | N | N | N | N |
| 0.3 ng/ml | N | N | N | N | N |
| 0.1 ng/ml | N | N | N | N | N |
| | 0 | 15 | 30 | 45 | 60 |
| Controls | minutes | minutes | minutes | minutes | minutes |
| 10 ng/ml intact cefotaxime | N | N | N | N | N |
| Extraction buffer 1X | P | P | P | P | P |

A signal is visible after 15 minutes incubation for 10 ng/ml of enzyme, and at 45 minutes for 3 ng/ml of enzymes. The same experiment was carried out, but this time with incubation at 37° C. (Table 7).

TABLE 7
Hydrolysis kinetics at 37° C. of intact cefotaxime by
CTXM-2, liquid tracer.

| Concentration of | 0 | 15 | 30 | 45 | 60 |
|---|---|---|---|---|---|
| CTXM-2 | minutes | minutes | minutes | minutes | minutes |
| 10 ng/ml | N | P | P | P | P |
| 3 ng/ml | N | N | N | P | P |
| 1 ng/ml | N | N | N | N | N |
| 0.3 ng/ml | N | N | N | N | N |
| 0.1 ng/ml | N | N | N | N | N |
| | 0 | 15 | 30 | 45 | 60 |
| Controls | minutes | minutes | minutes | minutes | minutes |
| 10 ng/ml intact | N | N | N | N | N |
| cefotaxime | | | | | |
| Extraction buffer | P | P | P | P | P |
| 1X | | | | | |

It can be seen in table 7 that incubation at 37° C. has no effect on the results. This is why incubation at ambient temperature was selected.

In order to limit the amount of handling and thus to facilitate use of the test, the tracer was dried on the CP. It was observed that after re-solubilising, part of the tracer antibody is absorbed by the CP. In order to compensate this absorption, the quantity of tracer used needed to be increased. The final tracer DO on the CP was 0.9 for this first test. The tests were carried out on 96-well microplates, with 100 μl of sample. The concentration of non-hydrolysed cefotaxime was 10 ng/ml. New hydrolysis kinetics were produced with this protocol (Table 8).

TABLE 8
Hydrolysis kinetics at AT for cefotaxime not hydrolysed by CTXM-2.

| Concentration | | | | | 60 |
|---|---|---|---|---|---|
| of CTXM-2 | 0 minutes | 15 minutes | 30 minutes | 45 minutes | minutes |
| 10 ng/ml | N | P | P | P | P |
| 3 ng/ml | N | P | P | P | P |
| 1 ng/ml | N | N | N | P | P |
| 0.3 ng/ml | N | N | N | N | N |
| 0.1 ng/ml | N | N | N | N | N |
| | | | | | 60 |
| Controls | 0 minutes | 15 minutes | 30 minutes | 45 minutes | minutes |
| 10 ng/ml | N | N | N | N | N |
| intact | | | | | |
| cefotaxime | | | | | |
| Extraction | P | P | P | P | P |
| buffer 1X | | | | | |

A signal was observed after 15 minutes at 3 ng/ml of enzymes and after 45 minutes at 1 ng/ml. The changing of the tracer to dried format and the increase in the DO made it possible to lower the concentration of enzymes necessary in order to see a visible signal on the TL.

With a view to always improving the performance of the test, two other hydrolysis kinetics were produced where the quantity of tracer in the CP was increased, being either 10 μl at DO:1 (Table 9), or 10 μl at DO:1.5 (Table 10). For this study, the strips were inserted in plastic cassettes and 100 μl of sample was deposited in the deposition wells. The concentration of non-hydrolysed cefotaxime was 10 ng/ml. The results were as follows.

TABLE 9
Hydrolysis kinetics of the intact cefotaxime by an enzyme
CTXM-2 (DO:1).

| Concentration | | | | | 60 |
|---|---|---|---|---|---|
| of CTXM-2 | 0 minutes | 15 minutes | 30 minutes | 45 minutes | minutes |
| 10 ng/ml | N | P | P | P | P |
| 3 ng/ml | N | P | P | P | P |
| 1 ng/ml | N | N | P | P | P |
| 0.3 ng/ml | N | N | N | N | N |
| 0.1 ng/ml | N | N | N | N | N |
| | | | | | 60 |
| Controls | 0 minutes | 15 minutes | 30 minutes | 45 minutes | minutes |
| 10 ng/ml | N | N | N | N | N |
| intact | | | | | |
| cefotaxime | | | | | |
| Extraction | P | P | P | P | P |
| buffer | | | | | |
| 1X | | | | | |

TABLE 10
Kinetics for hydrolysis of cefotaxime not hydrolysed by
CTXM-2 (DO1.5).

| Concentration of | 0 | | | | 60 |
|---|---|---|---|---|---|
| CTXM-2 | minutes | 15 minutes | 30 minutes | 45 minutes | minutes |
| 10 ng/ml | N | P | P | P | P |
| 3 ng/ml | N | P | P | P | P |
| 1 ng/ml | N | P | P | P | P |
| 0.3 ng/ml | N | N | N | N | N |
| 0.1 ng/ml | N | N | N | N | N |
| | 0 | | | | 60 |
| Controls | minutes | 15 minutes | 30 minutes | 45 minutes | minutes |
| 10 ng/ml | N | N | N | N | N |
| intact | | | | | |
| cefotaxime | | | | | |
| Extraction | P | P | P | P | P |
| buffer 1X | | | | | |

The increase in the DO in tracer made it possible to lower the concentration of enzymes necessary in order to have a visible signal on the TL. DO1.5 shows better results with detection at 1 ng/ml after 15 minutes.

The results obtained show that DO1.5 enabled a detection limit of 1 ng/ml of enzymes CTXM-2 after 15 minutes incubation.

Finalisation of the adaptation and validation of the test strip on bacterial colonies In order finalise the adaptation of the test strip and carry out its validation on bacterial colonies, two steps were performed:
Identification of the incubation time of non-hydrolysed cefotaxime with bacterial colonies in order to identify an ESBL type bacterial resistance.

In order to carry out these tests, all of the solutions below have been prepared in the buffer strip having, for composition: 0.1 M pH8 Tris/HCl buffer+0.15 M NaCl+0.5% Tween 20+1% Chaps+0.01% sodium azide, which makes it possible to lyse bacteria and release its contents.

The concentration of non-hydrolysed cefotaxime was 10 ng/ml in the buffer strip. Two controls were also produced containing, either only non-hydrolysed cefotaxime, or only the buffer strip. The tracer was dried on the CP. The TL was composed of 0.1 mg/ml of BSA-non-hydrolysed cefotaxime. The migration is carried out by plastic cassette.

150 μl of non-hydrolysed cefotaxime solution were sampled and deposited in an Eppendorf tube. A bacterial colony was sampled from an LB agar with a 1 μl ose, then deposited in the previously prepared Eppendorf tube. The tube was vortexed for five seconds, then incubated for a desired incubation time at ambient temperature. The samples were tested after 5 minutes; 10 minutes; 20 minutes; 30 minutes incubation. Once the incubation was finished, 100 μl was sampled and deposited on the strip. The reading was carried out after 10 minutes migration. A signal on the TL was considered to be “P”. An absence of signal on the TL was defined as “N”.

The results obtained are shown in table 11. Three groups have been defined: the group of ESBL, the group of carbapenemases (also degrade the intact cefotaxime), and the group of bacteria that are not resistant to cefotaxime.

TABLE 11
Determination of the incubation time at ambient temperature for
cefotaxime with bacterial colonies. N: negative result; P: positive result

| | | | | | |
|---|---|---|---|---|---|
| Enzyme | Species | 5′ | 10′ | 20′ | 30′ |

ESBL

| | | | | | |
|---|---|---|---|---|---|
| CTX-M-1 | C. freundii | P | P | P | P |
| CTX-M-2 | E. coli | P | P | P | P |
| CTX-M-8 | E. coli | N | N | P (low) | P |
| CTX-M-14 | K. oxytoca | N | P | P | P |
| CTX-M-15 | E. coli | P | P | P | P |
| CTX-M-27 | E. coli | P | P | P | P |
| CTX-M-32 | E. coli | P | P | P | P |
| CTX-M-57 | E. coli | P | P | P | P |
| CTX-M-94 | E. coli | P | P | P | P |

Carbapenemases

| | | | | | |
|---|---|---|---|---|---|
| KPC-2 | K. | N | P | P | P |
| | pneumoniae | | | | |
| NDM-7 | E. coli | P | P | P | P |
| IMP-1 | E. coli | P | P | P | P |
| VIM-1 | E. coli | P | P | P | P |
| OXA-48 | C. freundii | N | N | P (low) | P |
| OXA-163 | E. cloacae | P | P | P | P |

Sensitive bacteria

| | | | | | |
|---|---|---|---|---|---|
| WT | E. coli | N | N | N | N |
| WT | E. coli | N | N | N | N |

No signal was visible on the TL for the group of non-resistant bacteria after 30 minutes incubation. For the ESBL and carbapenemase groups, all of the bacterial colonies were detected after 20 minutes incubation, with the exception of two bacterial colonies which showed weak signals on the TL. At 30 minutes, all of the so-called resistant colonies were positive in our test.

In view of the results obtained, the incubation time of 30 minutes was selected. On applying this incubation time, all of the resistant colonies are positive and the non-resistant colonies are negative. The test strip is therefore well adapted to use on bacterial colonies.
Validation of the test strip on a strain of bacteria that is sensitive or resistant to third-generation cephalosporins (3GC).

In order to validate the test strip on bacterial colonies for its clinical use, a strain of bacteria resistant to cephalosporins was used.

In order to carry out these tests, all of the solutions below have been prepared in the buffer strip having, for composition: 0.1 M pH 8 Tris buffer/HCL+0.15 M NaCl+0.5% Tween 20+1% chaps+0.01% sodium azide. The concentration of non-hydrolysed cefotaxime used is 25 ng/ml in the buffer strip. The tracer is in dried format on the CP. The TL is composed of 0.1 mg/ml of BSA-non-hydrolysed cefotaxime. A CL was produced. It consists of anti-tracer antibodies. The migration is carried out in a plastic cassette.

In order to carry out the tests, 150 μl of non-hydrolysed cefotaxime solution was transferred into an Eppendorf tube. A bacterial colony was sampled from an URI-4 agar with a 1 μl ose, then deposited in the previously prepared Eppendorf tube. The tube was vortexed for five seconds, then incubated for 30 minutes at ambient temperature. Once the incubation was finished, 100 μl was sampled and deposited on the test strip. The reading is carried out after 10 minutes migration. A signal on the TL is considered as “P”. An absence of signal on the TL is defined as “N”.

In order to analyse the results, the bacteria were divided into two groups: β-lactamases producers which do not hydrolyse cefotaxime and β-lactamases producers which do hydrolyse cefotaxime (Table 12).

TABLE 12
Validation of the rapid test on bacterial colonies

| | | Isolate | | results of | |
|---|---|---|---|---|---|
| | Species | number | β-lactamase content | the test | |
| Strains not | E. coli | 1 | N/A | N | P |
| capable of | E. coli | 1 | MCR-1 | N | P |
| hydrolysing | E. coli | 1 | MCR-2 | N | P |
| cefotaxime | E. coli | 1 | MCR-5 | N | P |
| | E. coli | 1 | CTX-M-93 | N | P |
| | P. mirabilis | 1 | N/A | N | P |
| | P. mirabilis | 1 | OXA-23 | N | P |
| | P. mirabilis | 1 | OXA-58 | N | P |
| | C. freundii | 1 | N/A | N | P |
| | E. cloacae | 2 | N/A | N | P |
| | E. cloacae | 1 | IMI-1 | N | P |
| | E. asburiae | 2 | IMI-2 | N | P |
| | E. asburiae | 1 | IMI-17 | N | P |
| | Salmonella spp. | 1 | N/A | N | P |
| | Salmonella spp. | 1 | MCR-1 | N | P |
| | Salmonella spp. | 1 | MCR-4 | N | P |
| | Salmonella spp. | 1 | MCR-5 | N | P |
| | S. marcescens | 1 | SME-1 | N | P |
| | S. marcescens | 1 | SME-4 | N | P |
| | P. aeruginosa | 2 | N/A | N | P |
| | P. aeruginosa | 1 | Mex C/D-OprJ | N | P |
| | P. aeruginosa | 1 | Mex A/B-OprM | N | P |
| | P. aeruginosa | 1 | OprD deficient | N | P |
| | P. aeruginosa | 1 | CARBA-4 | N | P |
| | P. aeruginosa | 1 | OXA-32 | N | P |
| | P. aeruginosa | 1 | PME-1 | N | P |
| | P. aeruginosa | 1 | AIM-1 | N | P |
| | P. aeruginosa | 1 | OXA-198 | N | P |
| | P. putida | 1 | N/A | N | P |
| | P. stutzeri | 1 | N/A | N | P |
| | A. baumannii | 2 | N/A | N | P |
| | A. baumannii | 1 | RTG-4 | N | P |
| | A. baumannii | 1 | OXA-13 | N | P |
| | A. baumannii | 1 | OXA-21 | N | P |
| Strains | E. coli | 1 | Overexpressed AmpC | N | P |
| capable of | E. coli | 1 | DHA-1 | N | P |
| hydrolysing | E. coli | 1 | ACC-1 | N | P |
| cefotaxime | E. coli | 1 | CMY-136 | N | P |
| | E. coli | 1 | SHV-2a | N | P |
| | E. coli | 1 | TEM-52 | N | P |
| | E. coli | 3 | CTX-M-1 | N | P |
| | E. coli | 3 | CTX-M-2 | N | P |
| | E coli | 1 | CTX-M-3 | N | P |
| | E. coli | 1 | CTX-M-8 | N | P |
| | E. coli | 1 | CTX-M-10 | N | P |
| | E. coli | 2 | CTX-M-14 | N | P |
| | E. coli | 5 | CTX-M-15 | N | P |
| | E. coli | 1 | CTX-M-17 | N | P |
| | E. coli | 2 | CTX-M-24 | N | P |
| | E. coli | 2 | CTX-M-27 | N | P |
| | E. coli | 1 | CTX-M-32 | N | P |
| | E. coli | 1 | CTX-M-37 | N | P |
| | E. coli | 2 | CTX-M-55 | N | P |
| | E. coli | 1 | CTX-M-57 | N | P |
| | E. coli | 2 | CTX-M-65 | N | P |
| | E. coli | 1 | CTX-M-71 | N | P |
| | E. coli | 1 | CTX-M-82 | N | P |
| | E. coli | 1 | CTX-M-100 | N | P |
| | E. coli | 1 | CTX-M-101 | N | P |
| | E. coli | 1 | CTX-M-182 | N | P |
| | E. coli | 1 | KPC-2 | N | P |
| | E. coli | 1 | KPC-3 | N | P |
| | E. coli | 1 | KPC-5 | N | P |
| | E. coli | 1 | KPC-6 | N | P |
| | E. coli | 1 | KPC-7 | N | P |
| | E. coli | 1 | KPC-14 | N | P |
| | E. coli | 1 | KPC-28 | N | P |
| | E. coli | 1 | KPC-31 | N | P |
| | E. coli | 1 | KPC-33 | N | P |
| | E. coli | 1 | NDM-7 | N | P |
| | E. coli | 1 | NDM-19 | N | P |
| | E. coli | 1 | IMP-1 | N | P |
| | E. coli | 1 | IMP-14 | N | P |
| | E. coli | 2 | OXA-181 | N | P |
| | E. coli | 2 | OXA-244 | N | P |
| | E. coli | 1 | OXA-484 | N | P |
| | E. coli | 1 | ESBL + Mutation PmrB (G160E) | N | P |
| | E. coli | 1 | ESBL + MCR-1 | N | P |
| | E. coli | 1 | TEM-1 + OXA-244 | N | P |
| | E. coli | 1 | CMY + VIM-4 | N | P |
| | E. coli | 1 | CMY-13 + VIM-1 | N | P |
| | E. coli | 1 | CMY-2 + OXA-244 | N | P |
| | E. coli | 1 | SHV + DHA | N | P |
| | E. coli | 1 | SHV-12 + IMP-8 | N | P |
| | E. coli | 1 | CTX-M + MCR-3.2 | N | P |
| | E. coli | 1 | CTX-M-2 + MCR-1 | N | P |
| | E. coli | 1 | CTX-M-13 + CMY | N | P |
| | E. coli | 1 | CTX-M + NDM-19 | N | P |
| | E. coli | 1 | CTX-M-15 + OXA-48 | N | P |
| | E. coli | 1 | NDM-1 + MCR-1 | N | P |
| | E. coli | 2 | OXA-48 + MCR-1 | N | P |
| | E. coli | 1 | NDM-1 + VIM-2 | N | P |
| | E. coli | 1 | MCR-1 + OXA-48 + KPC-28 | N | P |
| | E. coli | 1 | TEM-1 + KPC-2 + OXA-9 | N | P |
| | E. coli | 1 | TEM-1 + NDM-9 + OXA-1 | N | P |
| | E. coli | 1 | CTX-M-9 + TEM-1 + KPC-2 | N | P |
| | E. coli | 1 | CTX-M-15 + CMY-6 + NDM-4 | N | P |
| | E. coli | 1 | CTX-M-15 + CMY-4 + OXA-204 | N | P |
| | E. coli | 1 | CTX-M + NDM-5 + OXA-181 | N | P |
| | E. coli | 1 | CTX-M-15 + NDM-6 + OXA-1 | N | P |
| | E. coli | 1 | CTX-M-15 + OXA-232 + OXA-1 | N | P |
| | E. coli | 1 | CTX-M-15 + CMY-2 + OXA-204 + | N | P |
| | | | OXA-1 | | |
| | E. coli | 1 | CTX-M-15 + CMY-4 + OXA-204 + | N | P |
| | | | OXA-1 | | |
| | E. coli | 1 | CTX-M-15 + TEM-1 + CMY + | N | P |
| | | | NDM-1 | | |
| | E. coli | 1 | CTX-M + SHV + NDM-1 + OXA- | N | P |
| | | | 48 | | |
| | E. coli | 1 | TEM-1 + CMY-16 + NDM-1 + | N | P |
| | | | OXA-1 + OXA-10 | | |
| | K. pneumoniae | 1 | DHA-2 | N | P |
| | K. pneumoniae | 1 | SHV-2a | N | P |
| | K. pneumoniae | 1 | SHV-11 | N | P |
| | K. pneumoniae | 1 | SHV-38 | N | P |
| | K. pneumoniae | 1 | TEM-3 | N | P |
| | K. pneumoniae | 1 | GES-1 | N | P |
| | K. pneumoniae | 1 | CTX-M-2 | N | P |
| | K. pneumoniae | 1 | CTX-M-3 | N | P |
| | K. pneumoniae | 1 | CTX-M-8 | N | P |
| | K. pneumoniae | 1 | CTX-M-15 | N | P |
| | K. pneumoniae | 1 | CTX-M-18 | N | P |
| | K. pneumoniae | 1 | CTX-M-19 | N | P |
| | K. pneumoniae | 1 | KPC-3 | N | P |
| | K. pneumoniae | 1 | NDM-1 | N | P |
| | K. pneumoniae | 1 | IMP-8 | N | P |
| | K. pneumoniae | 1 | OXA-48 | N | P |
| | K. pneumoniae | 2 | OXA-163 | N | P |
| | K. pneumoniae | 1 | OXA-517 | N | P |
| | K. pneumoniae | 1 | CMY-4 + OXA-204 | N | P |
| | K. pneumoniae | 1 | SHV-36 + TEM-1 | N | P |
| | K. pneumoniae | 2 | SHV-5 + VIM-1 | N | P |
| | K. pneumoniae | 1 | SHV-5 + IMP-1 | N | P |
| | K. pneumoniae | 1 | SHV-12 + IMP-8 | N | P |
| | K. pneumoniae | 1 | SHV-11 + OXA-48 | N | P |
| | K. pneumoniae | 2 | CTX-M + MCR-1 | N | P |
| | K. pneumoniae | 1 | CTX-M-15 + SHV-1 | N | P |
| | K. pneumoniae | 1 | CTX-M-15 + SHV-11 | N | P |
| | K. pneumoniae | 1 | CTX-M-15 + TEM | N | P |
| | K. pneumoniae | 1 | CTX-M + KPC-3 | N | P |
| | K. pneumoniae | 1 | CTX-M + OXA-370 | N | P |
| | K. pneumoniae | 1 | CTX-M + OXA-519 | N | P |
| | K. pneumoniae | 1 | KPC-4 + NDM-7 | N | P |
| | K. pneumoniae | 1 | KPC + VIM | N | P |
| | K. pneumoniae | 1 | CTX-M-15 + SHV-1 + TEM-1 | N | P |
| | K. pneumoniae | 1 | CTX-M-14 + SHV-11 + TEM-1 | N | P |
| | K. pneumoniae | 1 | CTX-M-15 + SHV-99 + TEM-1 | N | P |
| | K. pneumoniae | 1 | CTX-M + SHV-12 + GES-5 | N | P |
| | K. pneumoniae | 1 | CTX-M-15 + TEM-1 + OXA-48 | N | P |
| | K. pneumoniae | 1 | CTX-M + NDM-1 + OXA-232 | N | P |
| | K. pneumoniae | 1 | CTX-M + NDM-5 + OXA-232 | N | P |
| | K. pneumoniae | 1 | CTX-M + NDM-4 + KPC-2 | N | P |
| | K. pneumoniae | 1 | NDM-5 + VIM-1 + OXA-181 | N | P |
| | K. pneumoniae | 1 | MCR-1 + SHV-2 + SHV-106 + | N | P |
| | | | mgrB truncated in orf by IS5 | | |
| | K. pneumoniae | 1 | SHV-11 + TEM-1 + KPC-3 + | N | P |
| | | | OXA-9 | | |
| | K. pneumoniae | 1 | CTX-M-3 + SHV-1 + TEM-1 + | N | P |
| | | | VIM-19 | | |
| | K. pneumoniae | 1 | CTX-M + SHV-11 + TEM-1 + | N | P |
| | | | OXA-162 | | |
| | K. pneumoniae | 1 | CTX-M-15 + SHV-28 + TEM-1 + | N | P |
| | | | CMY + OXA-204 | | |
| | K. pneumoniae | 1 | CTX-M-15 + SHV-11 + TEM-1 + | N | P |
| | | | NDM-1 + OXA-1 | | |
| | K. pneumoniae | 1 | CTX-M-15 + SHV-1 + TEM-1 + | N | P |
| | | | OXA-232 + OXA-1 | | |
| | K. pneumoniae | 1 | CTX-M-15 + SHV-11 + TEM-1 + | N | P |
| | | | NDM-1 + OXA-1 + OXA-181 | | |
| | K. pneumoniae | 1 | CTX-M-15 + SHV-27 + TEM-1 + | N | P |
| | | | NDM-1 + OXA-1 + OXA-181 | | |
| | K. oxytoca | 2 | CTX-M-15 | N | P |
| | K. oxytoca | 1 | DHA-1 + SHV-75 | N | P |
| | P. mirabilis | 1 | ACC-1 | N | P |
| | P. mirabilis | 1 | TEM-52 | N | P |
| | P. mirabilis | 1 | CTX-M-71 | N | P |
| | C. freundii | 1 | SHV-12 | N | P |
| | C. freundii | 1 | CTX-M-1 | N | P |
| | C. freundii | 1 | CTX-M-15 | N | P |
| | C. freundii | 1 | CMY-150 + LMB-1 | N | P |
| | C. freundii | 2 | TEM-3 + Overexpressed AmpC | N | P |
| | C. freundii | 1 | TEM-1 + KPC-2 | N | P |
| | C. freundii | 1 | TEM-1 + VIM-2 | N | P |
| | C. freundii | 1 | SHV-12 + TEM-1 + OXA-48 | N | P |
| | C. freundii | 1 | CTX-M + VIM + OXA-48 | N | P |
| | C. freundii | 1 | CTX-M-15 + TEM-1 + CMY-48 + | N | P |
| | | | OXA-48 like | | |
| | C. freundii | 1 | CMY-135 + MOX-9 + OXA-372 + | N | P |
| | | | OXA-10 | | |
| | C. freundii | 1 | TEM-1 + VIM-2 + OXA-9 + | N | P |
| | | | OXA-10 | | |
| | C. koserii | 1 | CTX-M-1 | N | P |
| | C. koserii | 1 | TEM-1 + OXA-48 | N | P |
| | E. cloacae | 7 | Overexpressed AmpC | N | P |
| | E. cloacae | 1 | TEM-3 | N | P |
| | E. cloacae | 1 | GES-6 | N | P |
| | E. cloacae | 1 | CTX-M-9 | N | P |
| | E. cloacae | 1 | TMB-1 | N | P |
| | E. cloacae | 1 | GIM-1 | N | P |
| | E. cloacae | 1 | FRI-1 | N | P |
| | E. cloacae | 1 | IMI-3 + Overexpressed AmpC | N | P |
| | E. cloacae | 1 | NmcA + Overexpressed AmpC | N | P |
| | E. cloacae | 1 | SHV + GES-5 | N | P |
| | E. cloacae | 1 | CTX-M-15 + Overexpressed | N | P |
| | | | AmpC | | |
| | E. cloacae | 1 | SHV-5 + OXA-48 | N | P |
| | E. cloacae | 1 | SHV-12 + IMP-8 | N | P |
| | E. cloacae | 1 | SHV-70 + VIM-1 | N | P |
| | E. cloacae | 1 | SHV + OXA-163 | N | P |
| | E. cloacae | 1 | TEM-1 + KPC-2 | N | P |
| | E. cloacae | 1 | VIM-4 + OXA-48 | N | P |
| | E. cloacae | 1 | TEM-1 + KPC-2 + OXA-1 | N | P |
| | E. aerogenes | 2 | TEM-24 | N | P |
| | S. marcescens | 1 | OXA-405 | N | P |
| | S. marcescens | 1 | Overexpressed AmpC + | N | P |
| | | | Pénicillinase | | |
| | S. marcescens | 1 | ESBL + SME-2 | N | P |
| | S. marcescens | 1 | SHV-12 + IMP-10 | N | P |
| | S. marcescens | 1 | TEM-1 + KPC-2 | N | P |
| | M. morganii | 1 | Overexpressed AmpC | N | P |
| | M. morganii | 1 | CTX-M-15 | N | P |
| | S. enterica | 1 | CTX-M-15 + TEM-1 + OXA-1 + | N | P |
| | | | OXA-9 + OXA-10 + NDM-1 | | |
| | H. alvei | 2 | Overexpressed AmpC | N | P |
| | P. aeruginosa | 1 | SHV-2a | N | P |
| | P. aeruginosa | 1 | SHV-5 | N | P |
| | P. aeruginosa | 1 | TEM-4 | N | P |
| | P. aeruginosa | 1 | GES-2 | N | P |
| | P. aeruginosa | 1 | GES-5 | N | P |
| | P. aeruginosa | 1 | GES-9 | N | P |
| | P. aeruginosa | 1 | PER-1 | N | P |
| | P. aeruginosa | 1 | PER-2 | N | P |
| | P. aeruginosa | 1 | CTX-M-2 | N | P |
| | P. aeruginosa | 1 | BEL | N | P |
| | P. aeruginosa | 1 | VEB-1 | N | P |
| | P. aeruginosa | 1 | SPM-1 | N | P |
| | P. aeruginosa | 4 | KPC-2 | N | P |
| | P. aeruginosa | 2 | NDM-1 | N | P |
| | P. aeruginosa | 1 | VIM-1 | N | P |
| | P. aeruginosa | 2 | VIM-2 | N | P |
| | P. aeruginosa | 3 | VIM-4 | N | P |
| | P. aeruginosa | 1 | IMP-1 | N | P |
| | P. aeruginosa | 2 | IMP-2 | N | P |
| | P. aeruginosa | 1 | IMP-7 | N | P |
| | P. aeruginosa | 2 | IMP-13 | N | P |
| | P. aeruginosa | 1 | IMP-15 | N | P |
| | P. aeruginosa | 1 | IMP-19 | N | P |
| | P. aeruginosa | 1 | IMP-26 | N | P |
| | P. aeruginosa | 1 | IMP-29 | N | P |
| | P. aeruginosa | 1 | IMP-31 | N | P |
| | P. aeruginosa | 1 | IMP-39 | N | P |
| | P. aeruginosa | 1 | IMP-56 | N | P |
| | P. aeruginosa | 1 | IMP-63 | N | P |
| | P. aeruginosa | 1 | IMP-71 | N | P |
| | P. aeruginosa | 1 | GIM-1 + OXA-2 + OXA 395 | N | P |
| | P. aeruginosa | 1 | Overexpressed AmpC + OprD | N | P |
| | | | deficient + MexA/B-OprM | | |
| | P. aeruginosa | 1 | Overexpressed AmpC + OprD | N | P |
| | | | deficient + MexA/B-OprM + | | |
| | | | MexX/Y-OprM | | |
| | P. aeruginosa | 1 | Overexpressed AmpC + OprD | N | P |
| | | | deficient + MexA/B-OprM + | | |
| | | | MexC/D-OprJ | | |
| | P. fluorescens | 1 | VIM-2 | N | P |
| | P. putida | 1 | IMP-1 | N | P |
| | P. putida | 1 | IMP-46 | N | P |
| | P. putida | 1 | VIM-2 | N | P |
| | P. stutzeri | 1 | DIM-1 | N | P |
| | P. stutzeri | 1 | IMP-1 | N | P |
| | P. stutzeri | 1 | VIM-2 | N | P |
| | A. baumannii | 1 | SHV-5 | N | P |
| | A. baumannii | 1 | TEM | N | P |
| | A. baumannii | 1 | GES-11 | N | P |
| | A. baumannii | 1 | GES-12 | N | P |
| | A. baumannii | 3 | GES-14 | N | P |
| | A. baumannii | 1 | PER-1 | N | P |
| | A. baumannii | 1 | CTX-M-15 | N | P |
| | A. baumannii | 1 | SCO-1 | N | P |
| | A. baumannii | 1 | SIM-1 | N | P |
| | A. baumannii | 1 | VEB-1 | N | P |
| | A. baumannii | 1 | NDM | N | P |
| | A. baumannii | 2 | NDM-1 | N | P |
| | A. baumannii | 1 | NDM-2 | N | P |
| | A. baumannii | 2 | IMP-1 | N | P |
| | A. baumannii | 1 | IMP-4 | N | P |
| | A. baumannii | 1 | OXA-14 | N | P |
| | A. baumannii | 4 | OXA-23 | N | P |
| | A. baumannii | 1 | OXA-51 | N | P |
| | A. baumannii | 1 | OXA-58 | N | P |
| | A. baumannii | 1 | OXA-66 | N | P |
| | A. baumannii | 1 | OXA-72 | N | P |
| | A. baumannii | 1 | OXA-97 | N | P |
| | A. baumannii | 1 | OXA-143 | N | P |
| | A. baumannii | 1 | OXA-253 | N | P |
| | A. baumannii | 1 | Overexpressed AmpC + OXA-23 | N | P |
| | A. baumannii | 1 | SHV-5 + OXA-51 | N | P |
| | A. baumannii | 1 | GES-11 + OXA-23 | N | P |
| | A. baumannii | 1 | GES-11 + OXA-64 | N | P |
| | A. baumannii | 1 | GES-11 + OXA-98 | N | P |
| | A. baumannii | 3 | GES-12 + OXA-51 | N | P |
| | A. baumannii | 1 | GES-14 + OXA 23 | N | P |
| | A. baumannii | 5 | NDM-1 + OXA 23 | N | P |
| | A. baumannii | 1 | NDM-1 + OXA-51 | N | P |
| | A. baumannii | 1 | OXA-18 + OXA-20 | N | P |
| | A. baumannii | 1 | OXA-24 + OXA-40 | N | P |
| | A. baumannii | 1 | VEB-1 + OXA-10 + OXA-69 | N | P |
| | A. xylosoxidans | 1 | VIM-1 | N | P |
| | A. | 1 | VIM-4 | N | P |
| | genomospecies | | | | |

The 38 strains which cannot hydrolyse cefotaxime tested negative. There were therefore no false positives. Among the 300 strains which can hydrolyse cefotaxime, all the strains gave a signal on the TL.

Conclusion

Under the tested conditions, the strip test of the invention was able to obtain a sensitivity of 100% and a specificity of 100% for the detection of cephalosporinase activity in 40 minutes (incubation+migration). This performance is perfect for use in clinical and veterinary diagnosis and also in the context of environmental evaluation.

### EXAMPLE 2

### Detection of Bacteria Resistant to a Carbapenem

A. Design and Production of Immunogens
1) The alkyne carbapenem (S. Saidjalolov, Chem. Eur. J., 2021) is a small molecule incapable of inducing an immune response, essential for obtaining antibodies. It was therefore necessary to couple this antibiotic to a larger immunogenic molecule, bovine serum albumin (BSA). The production of the immunogen (carbapenem-BSA), called immunogen A, is carried out in two steps: step la consisted of producing BSA azide, then step 2a coupled the BSA azide with the alkyne carbapenem.

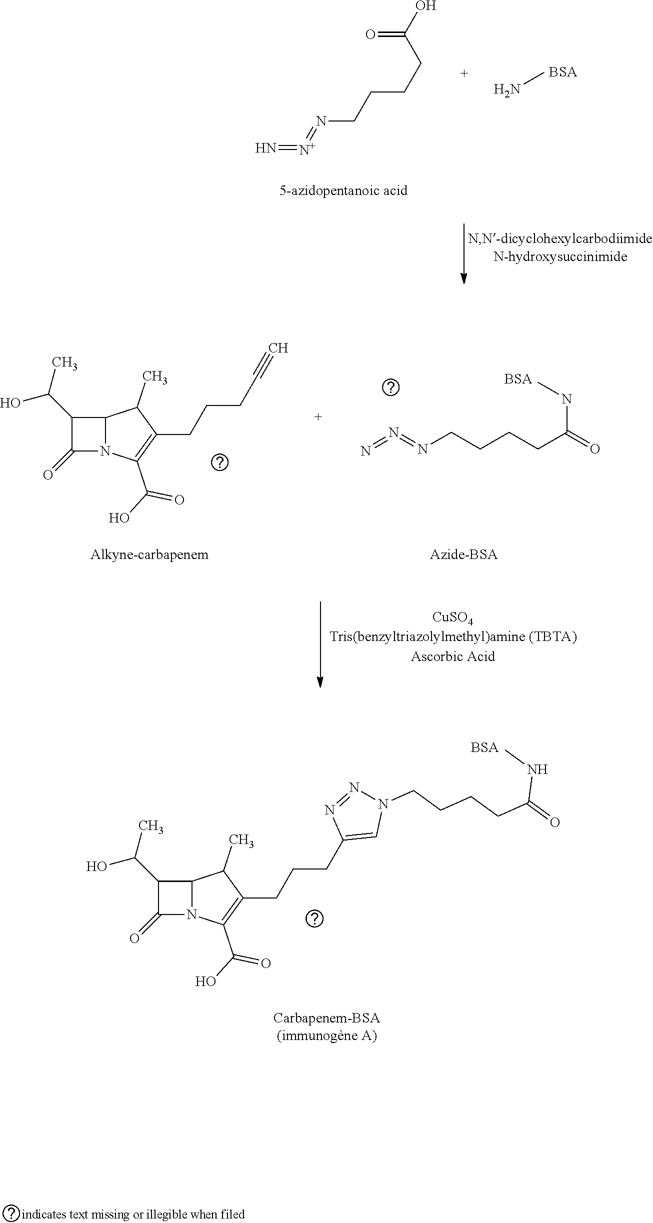

2) In parallel, another immunogen (carbapenem-BSA), called immunogen B, was produced by other chemical reactions. The production of this second immunogen took place in three steps: step 1a consisted of producing the SMC-BSA, then step 2a coupled the amine-carbapenem (Iannazzo L and al, 2016) with SATA (N-succinimidyl S-acetylthioacetate), and step 3a coupled SMC-BSA with SATA-carbapenem.

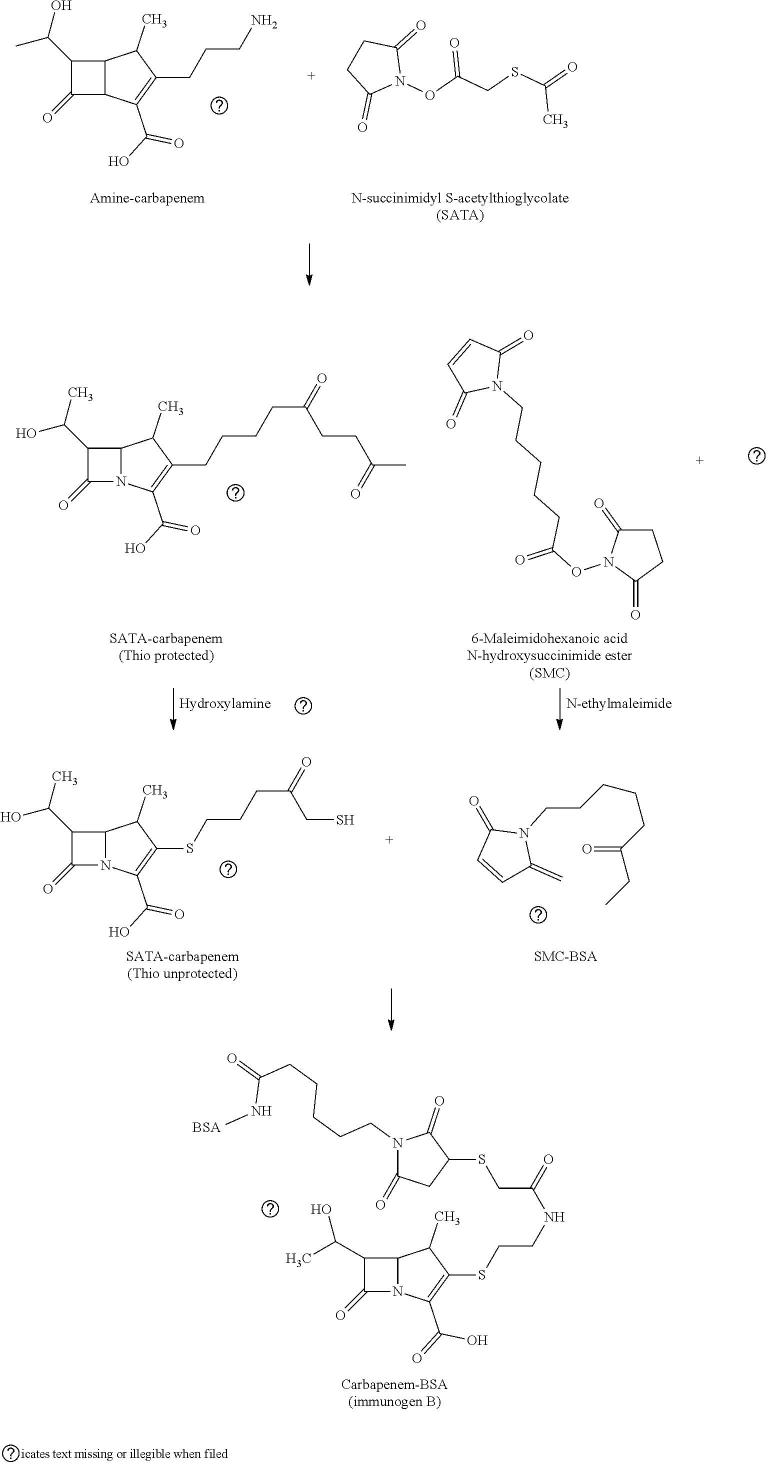

3) Immunogen A was used to immunise mice. In order to carry out the immunisations, subcutaneous injections of 50 μg of immunogen A/mouse were carried out every three weeks for three months (4 immunisations in total). After 2 months rest for the mice, new injections of immunogen A were carried out intravenously for said mice: 50 μg of product/mouse, once per day for three days. After two days rest, spleen cells of the mouse were fused with NS1 mouse myeloma cells, and anti-carbapenem specific antibodies in myeloma culture supernatants were detected using an immunoenzymatic test.

B. Production and Purification of the Various Forms of Carbapenem

For the proper implementation of the invention, it is essential that the difference in affinity of the antibodies of the invention for the intact and hydrolysed form of the antibiotic is maximal. To do this, it was necessary to have available non-hydrolysed carbapenem and hydrolysed carbapenem.

Production of Non-Hydrolysed and Hydrolysed Carbapenem-Biotin
1) Non-hydrolysed carbapenem-biotin was obtained by coupling amine-carbapenem and biotinamidohexanoic acid N-hydroxy-succinimide ester (NHS-LC-Biotin). The chemical reaction is produced between the NH₂ group of the amine carbapenem and the N-hydroxy-succinimide group of biotin. The resulting product is called tracer A.
2) Hydrolysed carbapenem-biotin(tracer A H) was obtained by enzymatic reaction with beads coupled with KPC-2 (Klebsiella pneumoniae carbapenemase), which is a recombinant β-lactamase. In order to do this, 50 μl of the solution of Beads-KPC-2 at 20 mg/ml were added to 1 ml of a solution of tracer A NH at 2 mg/ml. After reaction for 16 hours at 25° C., the Beads-KPC-2 were removed using a magnet. The supernatant containing the tracer A H was recovered and purified by reversed-phase chromatography. The molecular weight of this tracer was verified by mass spectrometry.

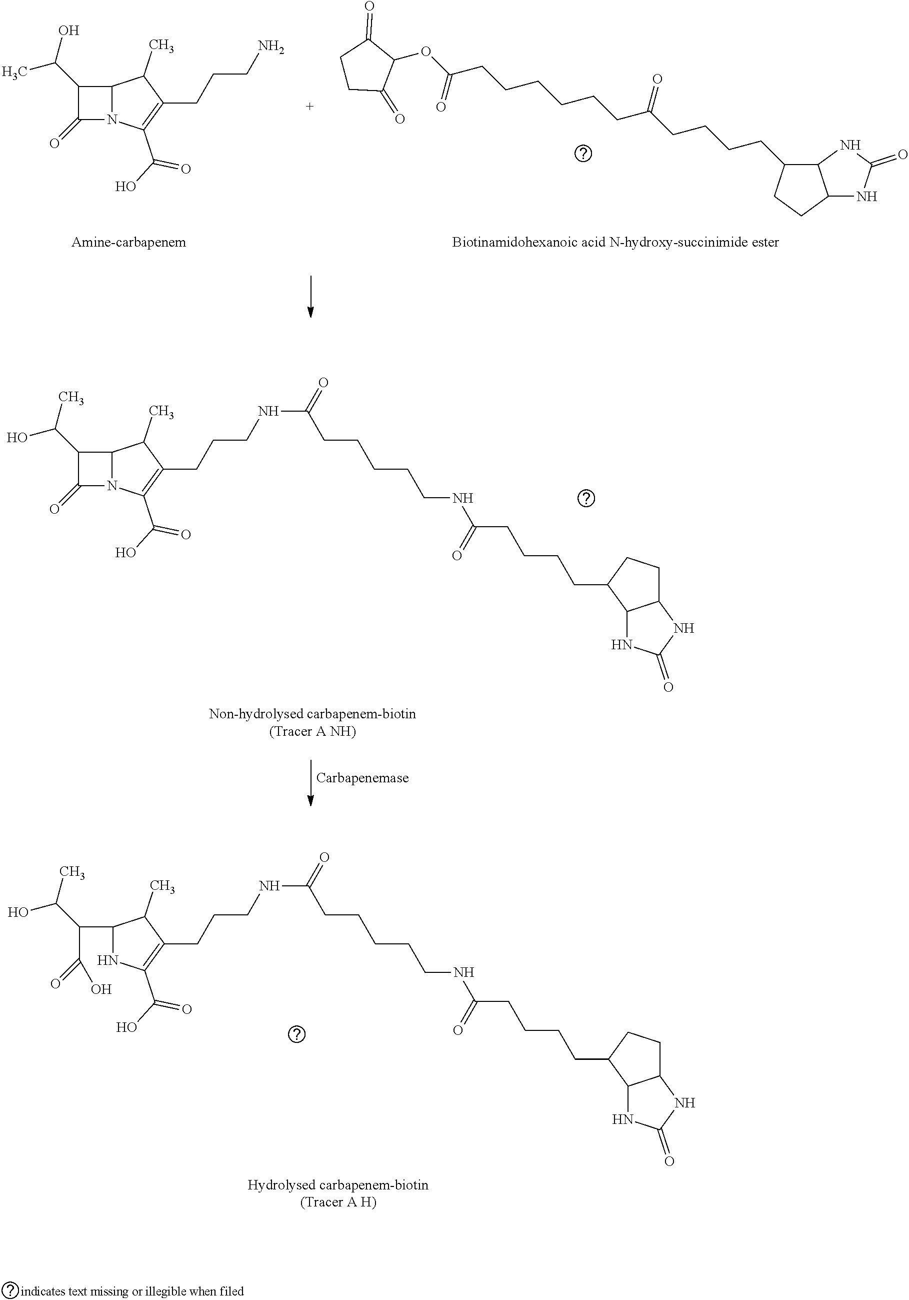 [SMILES: CC(O)C1C(=O)N2C(C(=O)O)=C(CCCN)C(C)C12 ; O=C(CCCCCCC(=O)OC1C(=O)CCC1=O)CCCCC1CCC2NC(=O)NC12 ; CC(O)C1C(=O)N2C(C(=O)O)=C(CCCNC(=O)CCCCCNC(=O)CCCCC3CCC4NC(=O)NC34)C(C)C12 ; CC(O)C(C(=O)O)C1NC(C(=O)O)=C(CCCNC(=O)CCCCCNC(=O)CCCCC2CCC3NC(=O)NC23)C1C]

3) In parallel, a second non-hydrolysed carbapenem-biotin was produced by another chemical process. It was produced by coupling an alkyne-carbapenem and biotin coupled to a polyethylene glycol (PEG) arm and an azide function (Biotin-dPEG®7-azide). The resulting product is called tracer B NH.
4) Hydrolysed carbapenem-biotin (tracer B H) was obtained by enzymatic reaction with beads coupled with KPC-2 (Klebsiella pneumoniae carbapenemase), which is a recombinant β-lactamase. In order to do this, 50 μl of the solution de Beads-KPC-2 at 20 mg/ml were added to 1 ml of a solution of tracer A NH at 2 mg/ml. After reaction for 16 hours at 25° C., the Beads-KPC-2 were removed using a magnet. The supernatant containing the tracer B H was recovered and purified by reversed-phase chromatography. The molecular weight of this tracer was verified by mass spectrometry.

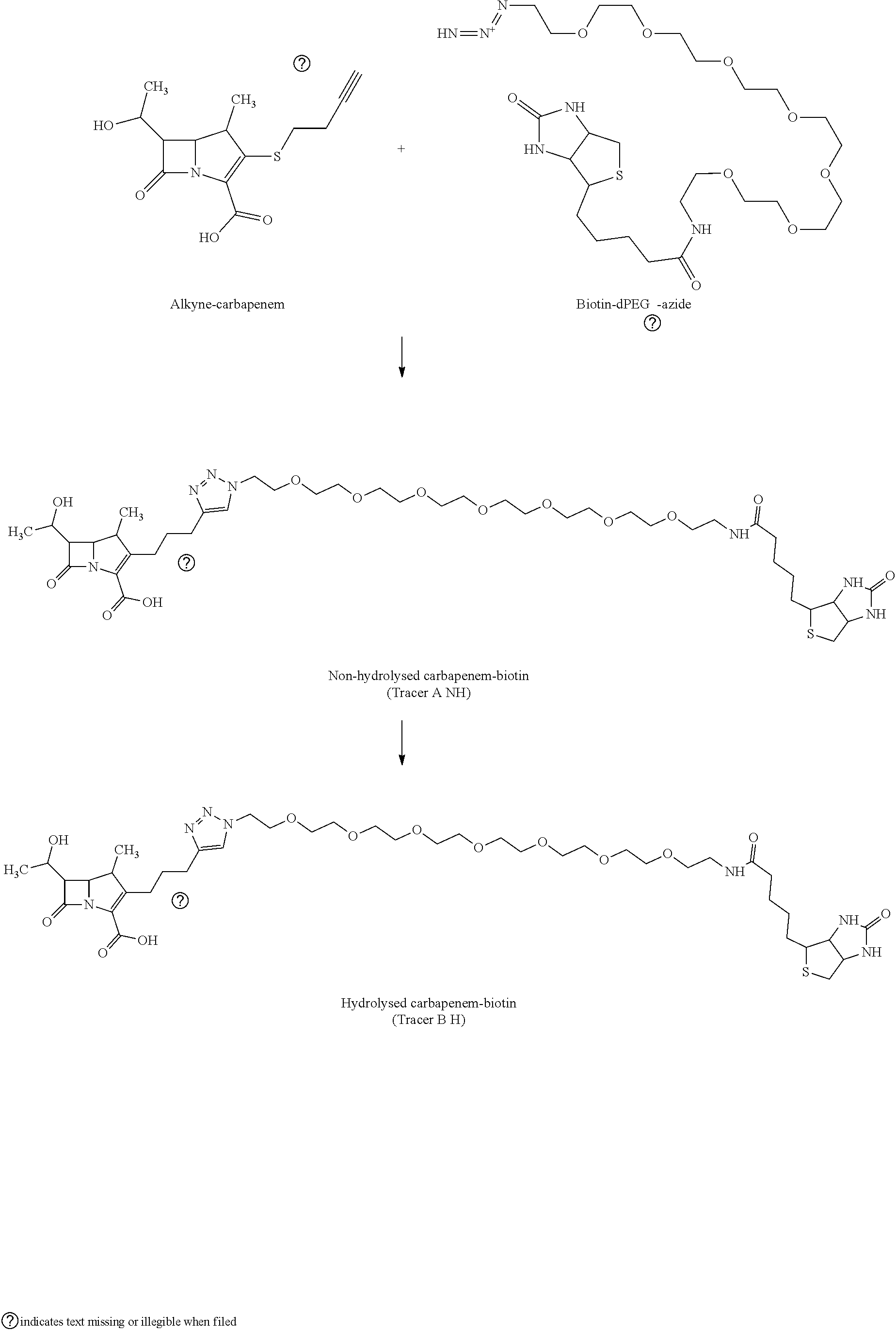 [SMILES: C#CCCSC1=C(C(=O)O)N2C(=O)C(C(C)O)C2C1C ; N=[N+]=NCCOCCOCCOCCOCCOCCOCCOCCNC(=O)CCCCC1SCC2NC(=O)NC21 ; CC(O)C1C(=O)N2C(C(=O)O)=C(CCCc3cn(CCOCCOCCOCCOCCOCCOCCOCCNC(=O)CCCCC4SCC5NC(=O)NC54)nn3)C(C)C12 ; CC(O)C1C(=O)N2C(C(=O)O)=C(CCCc3cn(CCOCCOCCOCCOCCOCCOCCOCCNC(=O)CCCCC4SCC5NC(=O)NC54)nn3)C(C)C12]

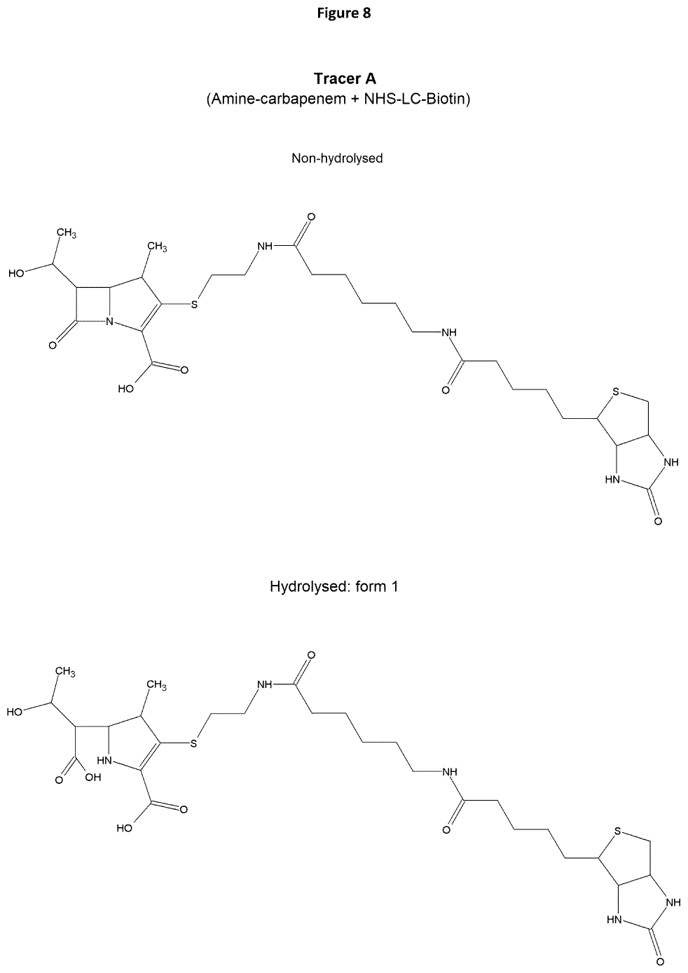

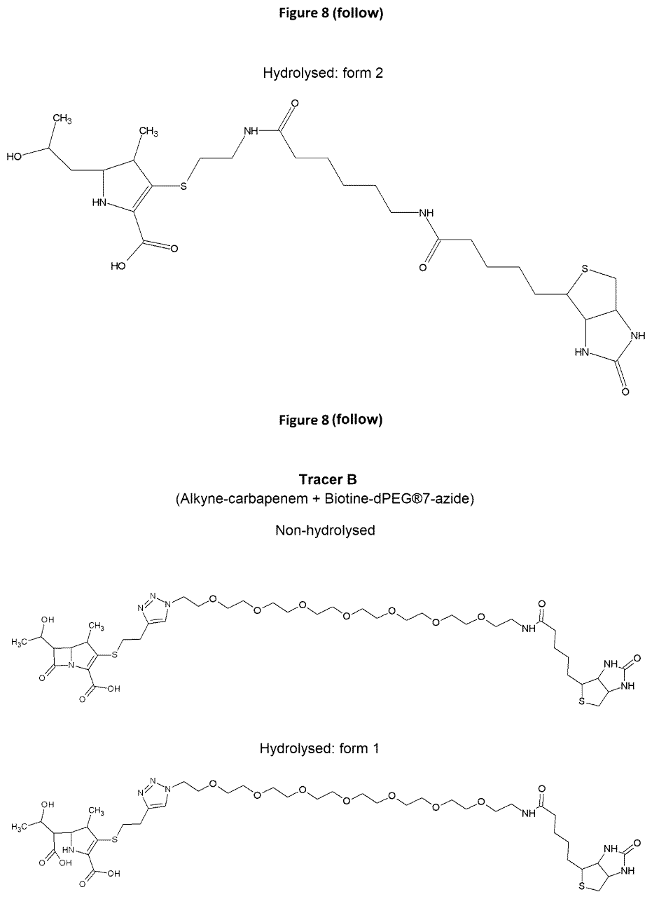

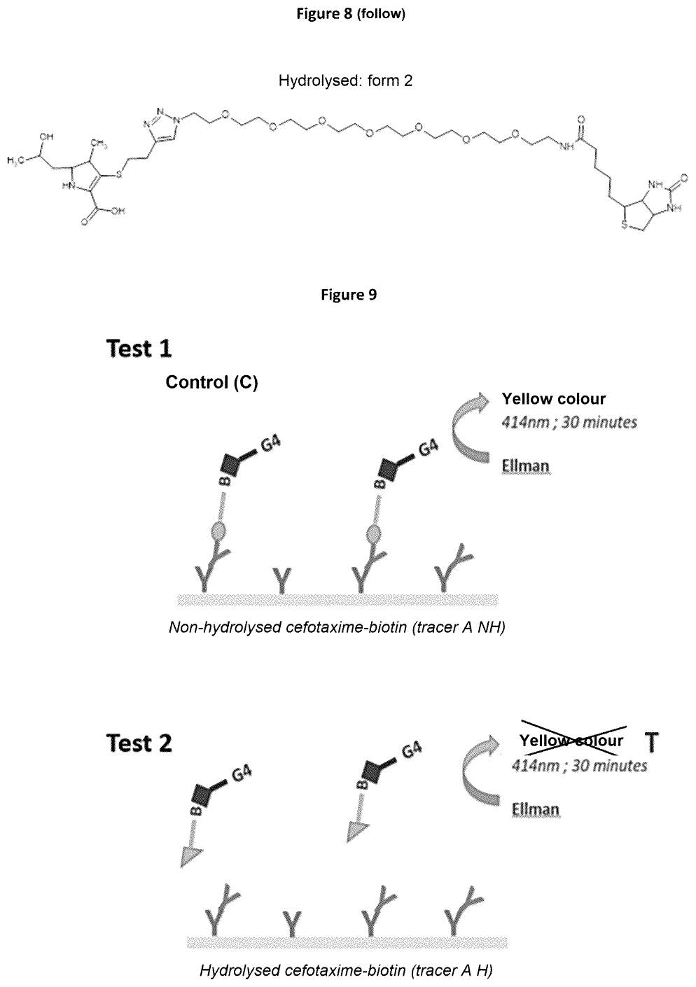

Production of Non-Hydrolysed and Hydrolysed Carbapenems
1) The non-hydrolysed carbapenem used for the selection of antibodies is meropenem (Sigma-Aldrich). The non-hydrolysed meropenem was purified by reversed-phase chromatography on a water/acetonitrile gradient of 0 to 20% (peak isolated at 7.8% acetonitrile).
2) The hydrolysed meropenem was also obtained by enzymatic reaction with beads coupled to KPC-2. The molecular weight of the molecule obtained was verified by mass spectrometry,
3) The same procedure was carried out on the four other carbapenems that are ertapenem, tebipenem, doripenem and imipenem.
4) The compounds described in FIG. 8 were used.

C. Production and Selection of the Antibodies of Interest

Four mice were immunised with immunogen A. In order to do this, subcutaneous injections of 50 μg of immunogen A/mouse were carried out every three weeks for three months (4 immunisations in total). After 3 months rest for the mice and in order to select the mice having the best immune response, their antibodies were analysed with a first test. In this test, the murine antibodies taken during the immunisation protocol were captured by a first murine anti-antibody antibody (AffiniPure Goat Anti-Mouse IgG+IgM (H+L); Jackson Immunoresearch LABORATORIES) immobilised on the wall of wells of a microtitration plate, by carrying out an incubation for 4 hours at ambient temperature under gentle stirring. After washing, 100 μL at 50 ng/ml of tracer A NH were added to each well and incubation took place overnight at 4° C. After washing, 100 μL de streptavidin-G4 at 1 EU/mL were added to reveal the presence of tracer A NH and therefore the presence of non-hydrolysed anti-carbapenem antibodies. Acetylcholinesterase (G4) activity was measured by the Ellman method (Ellman et al., 1961). The Ellman medium comprises a mixture of 7.5 10−4 M acetylthiocholine iodide (enzymatic substrate) and 2.5 10−4 M 5,5′-dithiobis(2-nitrobenzoic acid) (DTNB) (reagent for the calorimetry measurement of thiol) in a 0.1 M pH 7.4 phosphate buffer. The enzymatic activity is expressed in Ellman units (EU). One EU is defined as the quantity of enzyme producing an increase in absorbance of one unit during 1 minute in 1 ml of medium, for an optical path length of 1 cm: it corresponds to approximately 8 ng of enzyme.

After one hour incubation at ambient temperature and after washing, 200 μl of Ellman medium reagent are added to the wells. The signal intensities are measured after one hour. The intensity of the signals obtained during this test is then proportional to the quantity of tracer A NH specific antibodies. The mice having the best immune response (largest concentration of specific antibodies) received new intravenous injections of immunogen A: 50 μg of product/mouse, once per day for three days. After two days of rest, they were sacrificed and their splenocytes (spleen cells) were hybridised with NS1 mouse myeloma cells in order to obtain hybridomas.

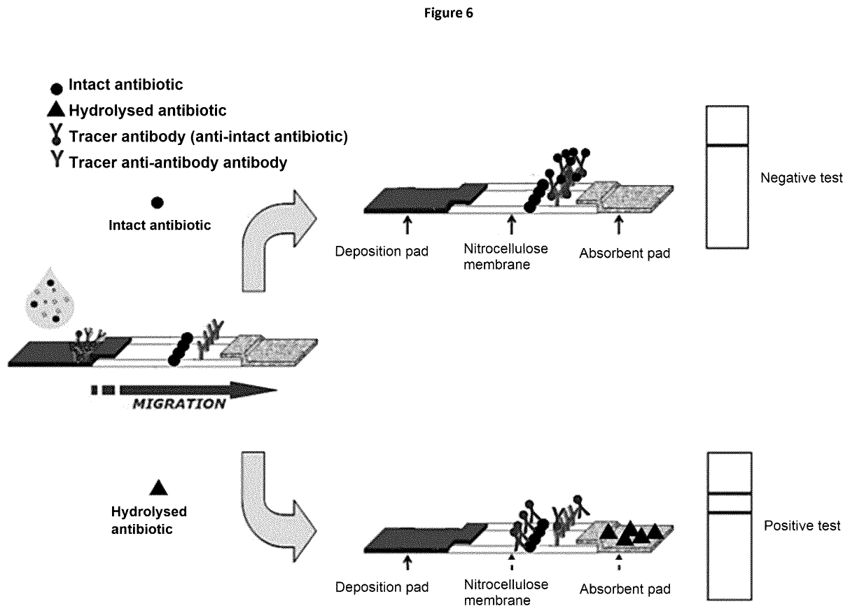

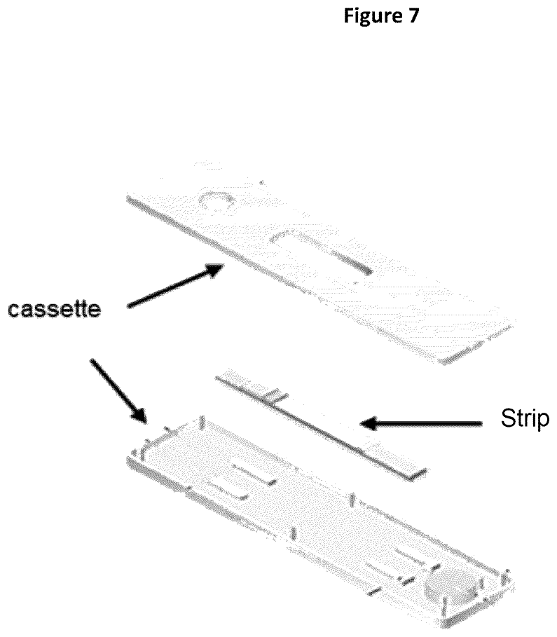

At the end of the fusion, all of the cells were distributed into the wells of 10 microtitration plates. After one week, the presence of antibodies recognising tracer A NH in each well was analysed using test 1 (FIG. 6). This test made it possible to select and preserve the cells from 93 wells producing non-hydrolysed anti-carbapenem antibodies. In order to refine this selection and keep the hybridomas producing antibodies specific to non-hydrolysed meropenem, the culture supernatants of the selected wells were analysed using 4 different tests (FIG. 6):

Test 1: In this test, the antibodies present in the culture supernatants are captured by a first murine anti-antibody antibody immobilised on the wall of wells of a microtitration plate. An incubation is carried out for 4 hours at ambient temperature under stirring. After washing, the tracer A NH-biotin is added to each well. After incubation at 4° C. overnight and after washing, streptavidin-G4 is added in order to reveal the presence of tracer A NH and therefore non-hydrolysed anti-cefotaxime antibodies.

Test 2: In this test, the antibodies present in the culture supernatants are captured by a first murine anti-antibody antibody immobilised on the wall of wells of a microtitration plate. An incubation is carried out for 4 hours at ambient temperature under stirring. After washing, tracer A H is added to each well. After incubation at 4° C. overnight and after washing, streptavidin-G4 is added in order to reveal the presence of tracer A H and therefore the presence of hydrolysed anti-cefotaxime antibodies.

Test 3: In this test, tracer A NH is placed in competition with non-hydrolysed meropenem with respect to recognition by the specific antibodies present in the culture supernatants. To do this, the antibodies present in the culture supernatants are captured by a first murine anti-antibody antibody immobilised on the wall of wells of a microtitration plate. An incubation is carried out for 4 hours at ambient temperature under stirring. After washing, tracer A NH and non-hydrolysed meropenem are added to each well. After incubation at 4° C. overnight and after washing, streptavidin-G4 is added in order to reveal the presence of tracer A NH.

Test 4: In this test, tracer A NH is placed in competition with hydrolysed meropenem at the same concentration as the non-hydrolysed meropenem used in test 3, with respect to recognition by the specific antibodies present in the culture supernatants. To do this, the antibodies present in the culture supernatants are captured by a first murine anti-antibody antibody immobilised on the wall of wells of a microtitration plate. An incubation is carried out for 4 hours at ambient temperature under stirring. After washing, tracer A NH and hydrolysed meropenem are added to each well. After incubation at 4° C. overnight and after washing, streptavidin-G4 is added in order to reveal the presence of tracer A NH.

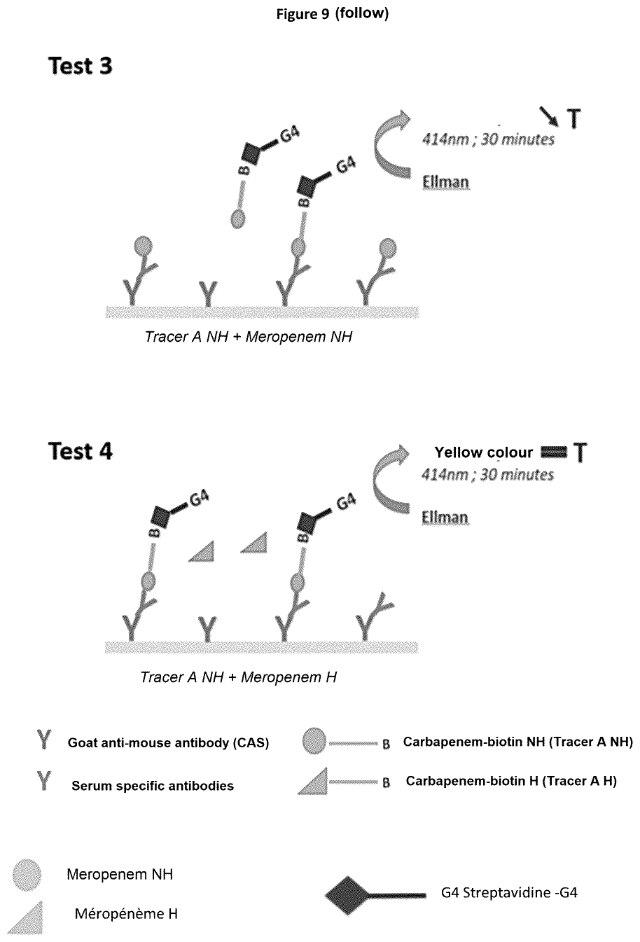

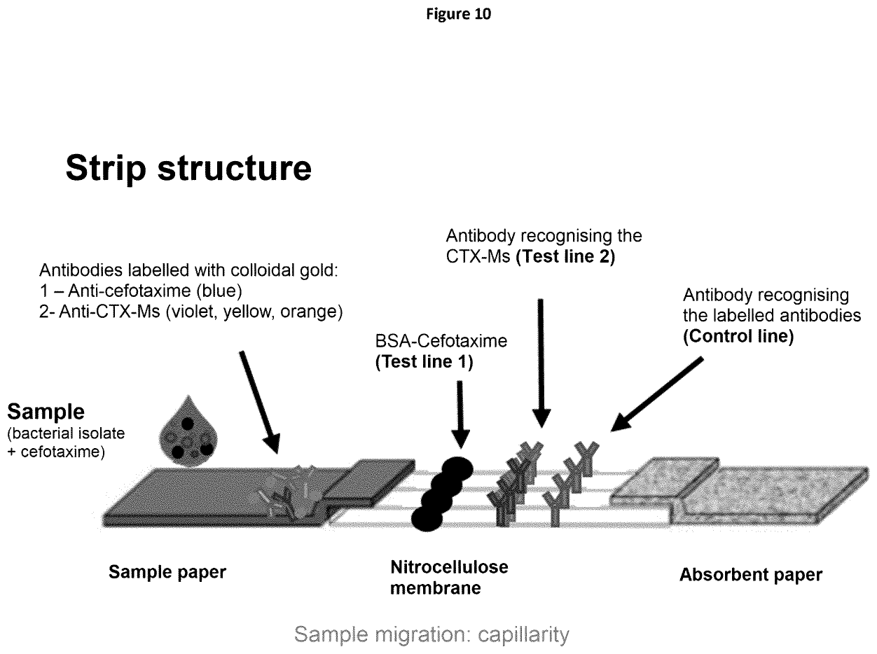

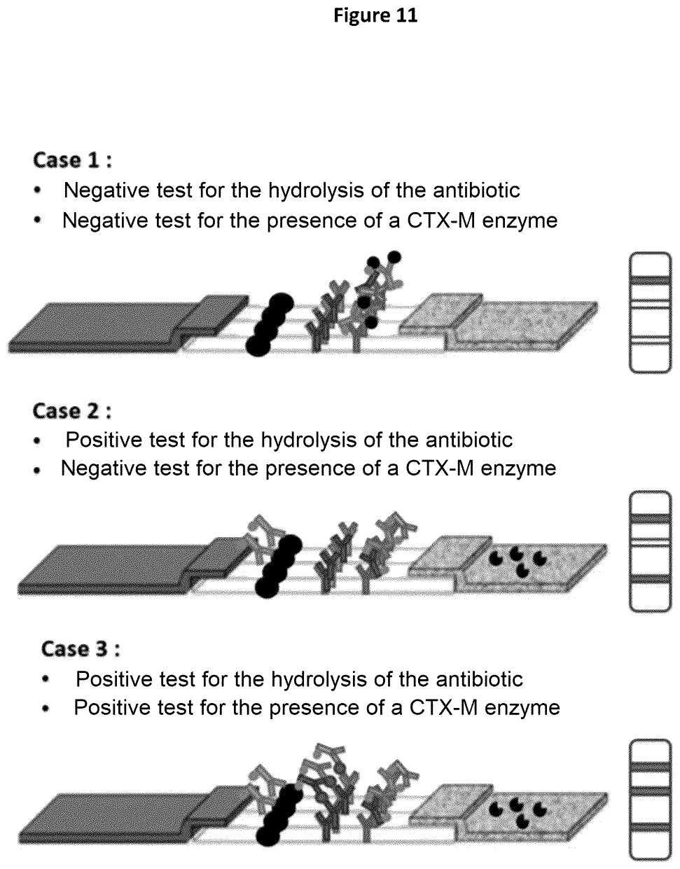

For tests 1 and 2, the appearance of the signal in the wells indicates the presence of non-hydrolysed anti-carbapenem antibodies and hydrolysed anti-carbapenem antibodies respectively (cf. FIG. 9).

For tests 3 and 4, a reduction in the signals proportional to the concentration of inhibitor reveals the presence of antibodies recognising the inhibitor: non-hydrolysed meropenem (test 3) or hydrolysed meropenem (test 4). These tests make it possible to evaluate the relative specificity of the antibodies for non-hydrolysed meropenem and hydrolysed meropenem. Hence, if the reduction in the signal is similar for the two forms of meropenem, the antibodies have the same affinity for these two molecules. If the reduction in the signal is weaker for one of the two forms of meropenem, then the antibodies have a weaker affinity for this form (FIG. 9).

The wells for which a signal is obtained for test 1 and no signal for test 2, a reduction in the largest signal of the signal for test 3 and no reduction of the signal for test 4, were selected. At the end of the selection process, 20 hybridomas were preserved in order to produce monoclonal antibodies.

### BIBLIOGRAPHICAL REFERENCES

Aguirre-Quiñonero, A., and Martinez-Martinez, L. (2017). Non-molecular detection of carbapenemases in Enterobacteriaceae clinical isolates. J. Infect. Chemother. 23, 1-11.
Bernabeu S, Ratnam K C, Boutal H, Gonzalez C, Vogel A, Devilliers K, Plaisance M, Oueslati S, Malhotra-Kumar S, Dortet L, Fortineau N, Simon S, Volland H, Naas T. (2020). A Lateral Flow Immunoassay for the Rapid Identification of CTX-M-Producing Enterobacterales from Culture Plates and Positive Blood Cultures. Diagnostics (Basel) 10.
Bonomo, R. A. (2017). β-Lactamases: A Focus on Current Challenges. Cold Spring Harb Perspect Med 7.
Boutal H, Vogel A, Bernabeu S, Devilliers K, Creton E, Cotellon G, Plaisance M, Oueslati S, Dortet L, Jousset A, Simon S, Naas T, Volland H. (2018). A multiplex lateral flow immunoassay for the rapid identification of NDM-, KPC-, IMP- and VIM-type and OXA-48-like carbapenemase-producing Enterobacteriaceae. J Antimicrob Chemother 73:909-915.
Cattoir, V. (2008). Les nouvelles β-lactamases à spectre étendu (BLSE). Pathologie infectieuse en réanimation 203-209.
Ellman L, Courtney D, Andres V, Featherstone R (1961) A new and rapid colorimetric determination of acetylcholinestare activity Biochemical Pharmacology 7 (2) 91-95
Iannazzo L, Soroka D, TribouleS, Fonvielle M, Compain F,. Dubée, J-Mainardi L, HugonneJ-E, Braud E, Arthur M, Etheve-Quelquejeu M, (2016) Routes of synthesis of carbapenems for optimizing both the inactivation of L,D-transpeptidase LdtMtl of Mycobacterium tuberculosis and the stability towards hydrolysis by β-lactamase BlaC. J. Med. Chem., 59, 7, 3427-3438
Iredell, J., Brown, J., and Tagg, K. (2016). Antibiotic resistance in Enterobacteriaceae: mechanisms and clinical implications. BMJ 352, h6420.
Lecour, A. (2017). Détection des carbapenemases chez les entérobactéries: revue de la littérature et évaluation des techniques phénotypiques. exercice. Université Toulouse III—Paul Sabatier.
Lutgring, J. D., and Limbago, B. M. (2016). The Problem of Carbapenemase-Producing-Carbapenem -Resistant-Enterobacteriaceae Detection. J. Clin. Microbiol. 54, 529-534.
Maugat, S., Berger-Carbonne, A., and Agence nationale de sécurité sanitaire de l'alimentation, de l'environnement et du travail (ANSES) (2018). Consommation d'antibiotiques et résistance aux antibiotiques en France: une infection évitée, c'est un antibiotique préservé!
Perez, F., Endimiani, A., Hujer, K. M., and Bonomo, R. A. (2007). The continuing challenge of ESBLs. Curr Opin Pharmacol 7, 459-469.
Public Health England (2019). Contained and controlled: the UK's 20-year vision for antimicrobial resistance.
S. Saidjalolov, E. Braud, Z. Edoo, L. Iannazzo, F. Rusconi, M. Riomet, A. Sallustrau, F. Taran, M. Arthur, M. Fonvielle, M. Etheve-Quelquejeu (2021) Click and Release Chemistry for Activity-based Purification of β-lactam targets. Chem. Eur. J., 2021

1. A method for detecting the presence of a functional β-lactamase enzyme in a sample, said method using at least one monoclonal antibody specifically recognising an antibiotic molecule containing an intact β-lactam ring, said antibody not recognising the same antibiotic molecule when hydrolysed, and the antibiotic molecule containing an intact β-lactam ring specifically recognised by said antibody.

2. The method according to claim 1, comprising the following steps:
a) placing said sample in contact with said antibiotic molecule containing an intact β-lactam ring,
b) placing said monoclonal antibody in contact with the sample obtained at the end of step a),
c) detecting whether said monoclonal antibody is complexed with said intact antibiotic.

3. The method according to claim 1, comprising the following steps, in this order:
a) placing the sample to be tested in contact with said antibiotic molecule containing an unlabelled intact β-lactam ring,
b) placing the sample obtained after step a) in contact with said antibody, which has been immobilised beforehand on a solid support,
c) placing said antibiotic molecule containing a labelled intact β-lactam ring, in contact with said antibody, or with the sample of step b) containing said antibody,
d) detecting whether said antibody is complexed with said antibiotic molecule added in step c).

4. The method according to claim 2, comprising the following steps, in this order:
a) placing the sample to be tested in contact with said antibiotic molecule containing an unlabelled intact β-lactam ring,
b) placing the sample obtained after step a) in contact with said antibody, which has been labelled,
c) placing the solution of step b) in contact with said antibiotic molecule containing an intact β-lactam ring, which has been immobilised on a solid support,
d) detecting whether said labelled antibody is complexed with the intact antibiotic placed in contact in step c).

5. The method according to claim 4, comprising the following steps, in this order:
a) placing the sample to be tested in contact with said antibiotic molecule containing an unlabelled intact β-lactam ring,
b) placing the sample obtained after step a) in contact with said antibody, and with at least one antibody specifically recognising a β-lactamase enzyme, said antibodies having been labelled,
c) placing the solution of step b) in contact with said antibiotic molecule containing an intact β-lactam ring and with antibodies specifically recognising a β-lactamase enzyme, which have been immobilised on a solid support,
d) detecting whether said labelled antibodies are complexed with the intact antibiotic and/or with the anti-β-lactamase antibodies, placed in contact in step c).

6. The method according to claim 1, wherein the sample contains bacteria.

7. The method according to claim 1, wherein said solid support is a strip.

8. The method according to claim 1, wherein said antibody specifically recognises cefotaxime or meropenem for which the β-lactam ring is intact.

9. A strip containing:
1) a zone for depositing the sample, on which at least one antibody specifically recognising an antibiotic molecule containing an intact β-lactam ring has been deposited, said antibody not recognising the same antibiotic molecule when hydrolysed, said antibody having been labelled beforehand,
2) a reaction zone, comprising:
at least one test line on which the antibiotic with intact β-lactam ring, having been used to produce at least one of the antibodies deposited in the deposition zone 1), has been immobilised, and
a control line on which antibodies recognising the antibodies present on the zone 1) have been immobilised,
and
3) an absorption zone promoting the migration of antibodies, said zone being situated at the opposite end of the deposition zone 1).

10. The strip of claim 9, on which antibodies recognising a β-lactamase enzyme have also been deposited and/or immobilised.

11. The strip of claim 10, containing:
the zone 1) for depositing the sample, on which have also been deposited antibodies recognising a β-lactamase enzyme, said antibodies having been labelled beforehand,
the reaction zone 2), further comprising at least one test line on which antibodies recognising a β-lactamase enzyme have been immobilised, the anti-β-lactamase antibodies deposited on the zone 1) and the anti-β-lactamase antibodies immobilised on the reaction zone 2).

12. The strip according to claim 9, wherein said antibodies specifically recognise cefotaxime or meropenem for which the β-lactam ring is intact.

13. A kit containing:
at least one strip as defined in claim 9, and
a separate container containing the antibiotic with intact β-lactam ring having been used to obtain the antibodies immobilised on the deposition zone 1) of the strip,
optionally, a separate container containing a β-lactamase enzyme and/or a sample not containing β-lactamase enzyme.

14. (canceled)

15. (canceled)

16. (canceled)

17. (canceled)

18. The method of claim 1, wherein said β-lactamase enzyme is an extended spectrum β-lactamase enzyme.

19. The strip of claim 10, wherein said antibodies recognising a β-lactamase enzyme have been deposited and/or immobilised on the deposition zone 1) and on a second test line in the reaction zone.

20. The strip of claim 11, wherein the anti-β-lactamase antibodies deposited on the zone 1) and the anti-β-lactamase antibodies immobilised on the reaction zone 2 detect different epitopes of the same β-lactamase enzymes.
